SEQUENCE LISTING (6 sequences)

SEQ ID NO: 1 - protein, 382 aa, Homo sapiens
MRSGGRGRPRLRLGERGLMEPLLPPKRRLLPRVRLLPLLLALAVGSAFYTIWSGWHRRTEELPLGRELRVPLIGSLPEARLRRVVGQLDPQRLWSTYLRPLLVVRTPGSPGNLQVRKFLEATLRSLTAGWHVELDPFTASTPLGPVDFGNVVATLDPRAARHLTLACHYDSKLFPPGSTPFVGATDSAVPCALLLELAQALDLELSRAKKQAAPVTLQLLFLDGEEALKEWGPKDSLYGSRHLAQLMESIPHSPGPTRIQAIELFMLLDLLGAPNPTFYSHFPRTVRWFHRLRSIEKRLHRLNLLQSHPQEVMYFQPGEPFGSVEDDHIPFLRRGVPVLHLISTPFPAVWHTPADTEVNLHPPTVHNLCRILAVFLAEYLGL

SEQ ID NO: 2 - DNA, 1149 bp, Homo sapiens
atgcgttccgggggccgcgggcgaccccgcctgcggctgggggaacgtggcctcatggagccactcttgccgccgaagcgccgcctgctaccgcgggttcggctcttgcctctgttgctggcgctggccgtgggctcggcgttctacaccatttggagcggctggcaccgcaggactgaggagctgccgctgggccgggagctgcgggtcccattgatcggaagcctccccgaagcccggctgcggagggtggtgggacaactggatccacagcgtctctggagcacttatctgcgccccctgctggttgtgcgaaccccgggcagcccgggaaatctccaagtcagaaagttcctggaggccacgctgcggtccctgacagcaggttggcacgtggagctggatcccttcacagcctcaacacccctggggccagtggactttggcaatgtggtggccacactggacccaagggctgcccgtcacctcacccttgcctgccattatgactcgaagctcttcccacccggatcgaccccctttgtaggggccacggattcggctgtgccctgtgccctgctgctggagctggcccaagcacttgacctggagctgagcagggccaaaaaacaggcagccccggtgaccctgcaactgctcttcttggatggtgaagaggcgctgaaggagtggggacccaaggactccctttacggttcccggcacctggcccagctcatggagtctatacctcacagccccggccccaccaggatccaggctattgagctctttatgcttcttgatctcctgggagcccccaatcccaccttctacagccacttccctcgcacggtccgctggttccatcggctgaggagcattgagaagcgtctgcaccgtttgaacctgctgcagtctcatccccaggaagtgatgtacttccaacccggggagccctctggctctgtggaagacgaccacatccccttcctccgcagaggggtacccgtgctccatctcatctccacgcccttccctgctgtctggcacacccctgcggacaccgaggtcaatctccacccacccacggtacacaacttgtgccgcattctcgctgtgttcctggctgaatacctggggctctag

SEQ ID NO: 3 - DNA, 19 bp, Artificial Sequence
  Synthetic Primer
ctcggcgttctacaccatt

SEQ ID NO: 4 - DNA, 18 bp, Artificial sequence
  Synthetic Primer
ctgctcagctccaggtca

SEQ ID NO: 5 - DNA, 28 bp, Artificial sequence
  Synthetic Primer
ctacaactggtcagcctgactactaacc

SEQ ID NO: 6 - DNA, 25 bp, Artificial sequence
  Synthetic Primer
tgatccaggaatctaaggcagcacc

(19) United States
(12) Patent Application Publication
(10) Pub. No.: US 2015/0150225 A1
(43) Pub. Date: Jun. 4, 2015

(54) TRANSGENIC MOUSE EXPRESSING HUMAN ISOGLUTAMINYL CYCLOTRANSFERASE

(71) Applicant: Probiodrug AG, Halle/Saale (DE)

(72) Inventors: Sigrid Graubner, Munchen (DE); Reinhard Sedlmeier, Munchen (DE); Andreas Becker, Munchen (DE); Stephan Schilling, Halle/Saale (DE); Holger Cynis, Halle/Saale (DE); Hans-Ulrich Demuth, Halle/Saale (DE)

(21) Appl. No.: 14/619,220

(22) Filed: Feb. 11, 2015

Related U.S. Application Data
(63) Continuation of application No. 13/225,033, filed on Sep. 2, 2011.
Application No. 13/225,033 is a continuation-in-part of application No. 12/016,266, filed on Jan. 18, 2008.
(60) Provisional application No. 60/885,649, filed on Jan. 19, 2007.

(51) Int. Cl. A01K 67/027; G01N 33/50; C12N 15/85; C12N 9/10

(52) U.S. Cl. CPC A01K 67/0278; C12N 9/104; G01N 33/5088; C12N 15/8509; C12Y 203/02005; A01K 2267/0312; A01K 2267/0318; A01K 2227/105; A01K 2217/052; A01K 2217/15; G01N 2333/9108; G01N 2500/10

(57) ABSTRACT

A transgenic non-human animal for overexpressing isoQC, comprising cells containing a DNA transgene encoding human isoQC, characterized in that said human isoQC comprises the amino acid sequence of SEQ ID NO: 1 or an amino acid sequence having at least 75% sequence identity to the amino acid sequence of SEQ ID NO: 1 or a fragment or derivative of the amino acid sequence of SEQ ID NO: 1. Additionally disclosed is a method of screening for biologically active agents that inhibit or promote isoQC.

### CROSS-REFERENCE TO RELATED APPLICATIONS

This is a Continuation of U.S. application Ser. No. 13/225,033, filed Sep. 2, 2011, which claims priority to U.S. application Ser. No. 12/016,266, filed Jan. 18, 2008, which claims priority to U.S. Provisional Application Ser. No. 60/885,649, filed Jan. 19, 2007. This application also claims priority to U.S. Provisional Application Ser. No. 61/379,451, filed Sep. 2, 2010. Each of the above references is incorporated herein by reference in its entirety to the extent permitted by law.

### MATERIAL INCORPORATED-BY-REFERENCE

The Sequence Listing, which is a part of the present disclosure, includes a computer readable form comprising nucleotide or amino acid sequences of the present invention. The subject matter of the Sequence Listing is incorporated herein by reference in its entirety.

### FIELD OF THE INVENTION

The present disclosure relates generally to transgenic animals as well as methods and compositions for screening and treating isoQC-related disorders, especially Alzheimer's disorder.

### BACKGROUND OF THE INVENTION

Glutaminyl cyclase (QC, EC 2.3.2.5; Qpct; glutaminyl peptide cyclotransferase) catalyzes the intramolecular cyclization of N-terminal glutamine residues into pyroglutamic acid (5-oxo-proline, pGlu*) under liberation of ammonia and the intramolecular cyclization of N-terminal glutamate residues into pyroglutamic acid under liberation of water.

A QC was first isolated by Messer from the Latex of the tropical plant Carica papaya in 1963 (Messer, M. 1963 Nature 4874, 1299). 24 years later, a corresponding enzymatic activity was discovered in animal pituitary (Busby, W. H. J. et al. 1987 J Biol Chem 262, 8532-8536; Fischer, W. H. and Spiess, J. 1987 Proc Natl Acad Sci USA 84, 3628-3632). For the mammalian QC, the conversion of Gln into pGlu by QC could be shown for the precursors of TRH and GnRH (Busby, W. H. J. et al. 1987 J Biol Chem 262, 8532-8536; Fischer, W. H. and Spiess, J. 1987 Proc Natl Acad Sci USA 84, 3628-3632). In addition, initial localization experiments of QC revealed a co-localization with its putative products of catalysis in bovine pituitary, further improving the suggested function in peptide hormone synthesis (Bockers, T. M. et al. 1995 J Neuroendocrinol 7, 445-453). In contrast, the physiological function of the plant QC is less clear. In the case of the enzyme from C. papaya, a role in the plant defense against pathogenic microorganisms was suggested (El Moussaoui, A. et al. 2001 Cell Mol Life Sci 58, 556-570). Putative QCs from other plants were identified by sequence comparisons recently (Dahl, S. W. et al. 2000 Protein Expr Purif 20, 27-36). The physiological function of these enzymes, however, is still ambiguous.

The QCs known from plants and animals show a strict specificity for L-glutamine in the N-terminal position of the substrates and their kinetic behavior was found to obey the Michaelis-Menten equation (Pohl, T. et al. 1991 Proc Natl Acad Sci USA 88, 10059-10063; Consalvo, A. P. et al. 1988 Anal Biochem 175, 131-138; Gololobov, M. Y. et al. 1996 Biol Chem Hoppe Seyler 377, 395-398). A comparison of the primary structures of the QCs from C. papaya and that of the highly conserved QC from mammals, however, did not reveal any sequence homology (Dahl, S. W. et al. 2000 Protein Expr Purif 20, 27-36). Whereas the plant QCs appear to belong to a new enzyme family (Dahl, S. W. et al. 2000 Protein Expr Purif 20, 27-36), the mammalian QCs were found to have a pronounced sequence homology to bacterial aminopeptidases (Bateman, R. C. et al. 2001 Biochemistry 40, 11246-11250), leading to the conclusion that the QCs from plants and animals have different evolutionary origins.

EP 02 011 349.4 discloses polynucleotides encoding insect glutaminyl cyclase, as well as polypeptides encoded thereby. This application further provides host cells comprising expression vectors comprising polynucleotides of the present disclosure. Isolated polypeptides and host cells comprising insect QC are useful in methods of screening for agents that reduce glutaminyl cyclase activity. Such agents are described as useful as pesticides.

The subject matter of the present disclosure is particularly useful in the field of isoQC-related diseases, one example of those being Alzheimer's Disease. Alzheimer's disease (AD) is characterized by abnormal accumulation of extracellular amyloidotic plaques closely associated with dystrophic neurones, reactive astrocytes and microglia (Terry, R. D. and Katzman, R. 1983 Ann Neurol 14, 497-506; Glenner, G. G. and Wong, C. W. 1984 Biochem Biophys Res Comm 120, 885-890; Intagaki, S. et al. 1989 J Neuroimmunol 24, 173-182; Funato, H. et al. 1998 Am J Pathol 152, 983-992; Selkoe, D. J. 2001 Physiol Rev 81, 741-766). Amyloid-beta (abbreviated as Aβ) peptides are the primary components of senile plaques and are considered to be directly involved in the pathogenesis and progression of AD, a hypothesis supported by genetic studies (Glenner, G. G. and Wong, C. W. 1984 Biochem Biophys Res Comm 120, 885-890; Borchelt, D. R. et al. 1996 Neuron 17, 1005-1013; Lemere, C. A. et al. 1996 Nat Med 2, 1146-1150; Mann, D. M. and Iwatsubo, T. 1996 Neurodegeneration 5, 115-120; Citron, M. et al. 1997 Nat Med 3, 67-72; Selkoe, D. J. 2001 Physiol Rev 81, 741-766). Aβ is generated by proteolytic processing of the β-amyloid precursor protein (APP) (Kang, J. et al. 1987 Nature 325, 733-736; Selkoe, D. J. 1998 Trends Cell Biol 8, 447-453), which is sequentially cleaved by β-secretase at the N-terminus and by γ-secretase at the C-terminus of Aβ (Haass, C. and Selkoe, D. J. 1993 Cell 75, 1039-1042; Simons, M. et al. 1996 J Neurosci 16 899-908). In addition to the dominant Aβ peptides starting with L-Asp at the N-terminus (Aβ1-42/40), a great heterogeneity of N-terminally truncated forms occurs in senile plaques. Such shortened peptides are reported to be more neurotoxic in vitro and to aggregate more rapidly than the full-length isoforms (Pike, C. J. et al. 1995 J Biol Chem 270, 23895-23898). N-truncated peptides are known to be overproduced in early onset familial AD (FAD) subjects (Saido, T. C. et al. 1995 Neuron 14, 457-466; Russo, C, et al. 2000 Nature 405, 531-532), to appear early and to increase with age in Down's syndrome (DS) brains (Russo, C. et al. 1997 FEBS Lett 409, 411-416, Russo, C. et al. 2001 Neurobiol Dis 8, 173-180; Tekirian, T. L. et al. 1998 J Neuropathol Exp Neurol 57, 76-94). Finally, their amount reflects the progressive severity of the disease (Russo, C. et al. 1997 FEBS Lett 409, 411-416; Guntert, A. et al. 2006 Neuroscience 143, 461-475). Additional post-translational processes may further modify the N-terminus by isomerization or racemization of the aspartate at position 1 and 7 and by cyclization of glutamate at residues 3 and 11. Pyroglutamate-containing isoforms at position 3 [pGlu³Aβ3-40/42] represent the prominent forms—approximately 50% of the total Aβ amount—of the N-truncated species in senile plaques (Mori, H. et al. 1992 J Biol Chem 267, 17082-17086, Saido, T. C. et al. 1995 Neuron 14, 457-466; Russo, C. et al. 1997 FEBS Lett 409, 411-416; Tekirian, T. L. et al. 1998 J Neuropathol Exp Neurol 57, 76-94; Geddes, J. W. et al. 1999 Neurobiol Aging 20, 75-79; Harigaya, Y. et al. 2000 Biochem Biophys Res Commun 276, 422-427) and they are also present in pre-amyloid lesions (Lalowski, M. et al. 1996 J Biol Chem 271, 33623-33631). The accumulation of AβN3(pE) peptides is likely due to the structural modification that enhances aggregation and confers resistance to most aminopeptidases (Saido, T. C. et al. 1995 Neuron 14, 457-466; Tekirian, T. L. et al. 1999 J Neurochem 73, 1584-1589). This evidence provides clues for a pivotal role of AβN3(pE) peptides in AD pathogenesis. However, relatively little is known about their neurotoxicity and aggregation properties (He, W. and Barrow, C. J. 1999 Biochemistry 38, 10871-10877; Tekirian, T. L. et al. 1999 J Neurochem 73, 1584-1589). Moreover, the action of these isoforms on glial cells and the glial response to these peptides are completely unknown, although activated glia is strictly associated with senile plaques and might actively contribute to the accumulation of amyloid deposits. In recent studies the toxicity, aggregation properties and catabolism of Aβ1-42, Aβ1-40, [pGlu³]Aβ3-42, [pGlu³]Aβ3-40, [pGlu¹¹]Aββ11-42 and [pGlu¹¹]Aβ11-40 peptides were investigated in neuronal and glial cell cultures, and it was shown that pyroglutamate modification exacerbates the toxic properties of Aβ-peptides and also inhibits their degradation by cultured astrocytes. Shirotani et al. investigated the generation of [pGlu³]Aβ peptides in primary cortical neurons infected by Sindbis virus in vitro. They constructed amyloid precursor protein complementary DNAs, which encoded a potential precursor for [pGlu³]Aβ by amino acid substitution and deletion. For one artificial precursor starting with a N-terminal glutamine residue instead of glutamate in the natural precursor, a spontaneous conversion or an enzymatic conversion by glutaminyl cyclase to pyroglutamate was suggested. The cyclization mechanism of N-terminal glutamate at position 3 in the natural precursor of [pGlu³]Aβ was neither determined in vitro, in situ nor in vivo (Shirotani, K. et al. 2002 NeuroSci Lett 327, 25-28).

Isoenzymes of QC (i.e. isoglutaminyl peptide cyclotransferase; isoQC; QPCTL) have been described in WO 2008/034891, WO 2008/087197 and WO 2010/026209 (each in the name of Probiodrug AG). Accordingly, it is an object of the present disclosure to provide a transgenic animal, which overexpresses isoQC. It is another object of the present disclosure to provide DNA constructs encoding isoQC. It is an additional object of the present disclosure to provide DNA constructs encoding isoQC linked to a promoter. It is an additional object of the present disclosure to provide a non-human transgenic animal model system to study the in vivo and in vitro regulation and effects of isoQC in specific tissue types.

### SUMMARY OF THE INVENTION

The present disclosure provides methods and compositions for non-human transgenic, in particular mammal, models for isoQC-related diseases. Specifically, the present disclosure provides non-human transgenic animal models that overexpress isoQC.

The present disclosure further provides compositions and methods for screening for biologically active agents that modulate isoQC-related diseases including, but not limited to, Mild Cognitive Impairment (MCI), Alzheimer's Disease (AD), cerebral amyloid angiopathy, Lewy body dementia, neurodegeneration in Down Syndrome, hereditary cerebral hemorrhage with amyloidosis (Dutch type), Familial Danish Dementia, Familial British Dementia, ulcer disease and gastric cancer with or w/o Helicobacter pylori infections, pathogenic psychotic conditions, schizophrenia, infertility, neoplasia, inflammatory host responses, cancer, psoriasis, rheumatoid arthritis, atherosclerosis, restenosis, lung fibrosis, liver fibrosis, renal fibrosis, Acquired Immune Deficiency Syndrome, graft rejection, Chorea Huntington (HD), impaired humoral and cell-mediated immune responses, leukocyte adhesion and migration processes in the endothelium, impaired food intake, sleep-wakefulness, impaired homeostatic regulation of energy metabolism, impaired autonomic function, impaired hormonal balance and impaired regulation of body fluids and the Guam Parkinson-Dementia complex. Another embodiment of the present disclosure provides methods and compositions for screening for isoQC inhibitors.

Further, by administration of effectors of isoQC activity to a mammal it can be possible to stimulate gastrointestinal tract cell proliferation, preferably proliferation of gastric mucosal cells, epithelial cells, acute acid secretion and the differentiation of acid producing parietal cells and histamine-secreting enterochromaffin-like cells.

Furthermore, by administration of effectors of isoQC activity to a mammal it can be possible to suppress the proliferation of myeloid progenitor cells.

In addition, administration of isoQC inhibitors can lead to suppression of male fertility.

The present disclosure provides pharmaceutical compositions for parenteral, enteral or oral administration, comprising at least one effector of isoQC optionally in combination with customary carriers or excipients.

Additionally, the present disclosure provides methods and compositions for the treatment or prevention of isoQC-related diseases, particularly methods and compositions that inhibit or promote isoQC.

It was shown by inhibition studies that human and murine QC are metal-dependent transferases. QC apoenzyme could be reactivated most efficiently by zinc ions, and the metal-binding motif of zinc-dependent aminopeptidases is also present in human QC. Compounds interacting with the active-site bound metal are potent inhibitors.

Unexpectedly, it was shown that recombinant human QC as well as QC-activity from brain extracts catalyze both, the N-terminal glutaminyl as well as glutamate cyclization. Most striking is the finding, that QC-catalyzed Glu¹-conversion is favored around pH 6.0 while Gln¹-conversion to pGlu-derivatives occurs with a pH-optimum of around 8.0. Since the formation of pGlu-Aβ-related peptides can be suppressed by inhibition of recombinant human QC and QC-activity from pig pituitary extracts, the enzyme QC is a target in drug development for treatment of e.g. Alzheimer's disease.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1: tgisoQC expression cassette

For expression of the hisoQC in the transgenic mouse line, the hisoQC coding sequence was fused upstream with the mouse Thy1 promoter region comprising the promoter and the 5′-untranslated region including exon 1 and exon 2 of the Thy 1 gene. In addition, the hisoQC coding sequence was fused downstream to the 3′-untranslated Thy1 region containing the polyadenylation signal.

FIG. 2: Genotyping strategies for PCR detection of the hisoQC transgene

PCR1: PCR using primer pairs tgisoQC-3 (SEQ ID NO: 3) and -4 (SEQ ID NO: 4) and chromosomal DNA from hisoQC-transgenic animals delivers a 486 bp PCR product, which is indicative of the presence of the transgene expression cassette in the chromosome.
PCR2: PCR using primer pairs GX3626 (SEQ ID NO: 5) and GX3627 (SEQ ID NO: 6) and chromosomal DNA from hisoQC-transgenic animals delivers a 7097 bp PCR product, which is indicative of the integrity of the transgene expression cassette in the chromosome.

FIG. 3 shows immunohistochemical staining of coronal sections of the hippocampus of wildtype, QPCTL knockout, and tgisoQC-31 heterozygous mice with isoQC antibody (scale bars: 500 μm).

FIG. 4 shows immunohistochemical staining of coronal sections of the hippocampal CA1 region of wildtype, tgisoQC-13 heterozygous, tgisoQC-20 heterozygous, and tgisoQC-31 mice with NeuN and GFAP antibodies (scale bars: 50 μm).

FIG. 5 shows immunohistochemical staining of coronal sections of the hippocampus of wildtype, QPCTL knockout, and tgisoQC-31 heterozygous mice with GFAP antibody (scale bars: 100 μm).

FIG. 6 shows immunohistochemical staining of coronal sections of the hippocampal CA1 region of wildtype, QPCTL knockout, and tgisoQC-31 heterozygous mice with lba1 antibody (scale bars: 200 μm).

FIG. 7 shows double immunofluorescence staining of coronal sections of the hippocampal CA1 region of wildtype, tgisoQC-13 heterozygous, tgisoQC-20 heterozygous and tgisoQC-31 heterozygous mice with NeuN (red) and GFAP (green) antibodies (scale bars: 200 μm).

FIG. 8 shows the results of a determination of isoQC activity in isoQC-transgenic and wild-type mice. All transgenic mice showed a significant overexpression of isoQC, as shown by the increase of activity compared to wild type mice.

FIG. 9 shows locomotor activity in the x/y-level of wildtype and heterozygous tgisoQC mice in the automated home cage behavior analysis using a PhenoMaster system. (a) Total distance moved during a 136 hour investigation period shown as mean+SEM (**, p<0.01, t-test) and (b) locomotor activity patterns during 12 hour light/12 hour dark (gray bars) cycles shown as mean of sum over 1 hour intervals.

FIG. 10 shows rearing activity in the z-level of wildtype and heterozygous tgisoQC mice in the automated home cage behavior analysis using a PhenoMaster system. (a) Total rearing activity during a 136 hour investigation period shown as mean+SEM (***, p<0.001, t-test) and (b) vertical activity patterns during 12 hour light/12 hour dark (gray bars) cycles shown as mean of sum over 1 hour intervals.

FIG. 11 shows ingestion behavior of wildtype and heterozygous tgisoQC mice in the automated home cage behavior analysis using a PhenoMaster system. (a) Total water consumption and (b) total food consumption during a 136 hour investigation period shown as mean+SEM.

FIG. 12 shows the duration of stay in the light compartment (mean+SEM, **, p<0.01, t-test) of wildtype and heterozygous tgisoQC female mice aged 2.5 months during a dark-light box test.

FIG. 13 shows the weight course of heterozygous tgisoQC females and wildtype littermates consisting of data collected within the primary screen at three stages of life (mean+SEM).

FIG. 14 shows performance of wildtype and heterozygous tgisoQC females aged 2.5 months on the pole as (a) time to turn around (t-turn) and (b) total time to climb down (t-total) in the best out of five trials (mean+SEM).

FIG. 15 shows performance of wildtype and heterozygous tgisoQC females aged 3 months on the accelerating rotarod (4 to 40 rpm in 300 seconds) as total distance moved (mean+SEM): (a) best trial analysis out of nine trials, (b) trial progression.

FIG. 16 shows the results of the holeboard test of wildtype and heterozygous tgisoQC female mice aged 3 months: (a) number of nosepokes and (b) total duration of hole exploration are shown as mean+SEM (*, p<0.05, t-test).

FIG. 17 shows the tail withdrawal latency (mean+SEM) in a tail flick test of wildtype and heterozygous tgisoQC female mice aged 3 months.

FIG. 18 shows the paw withdrawal latency of wildtype and heterozygous tgisoQC females on the constant hotplate (52.5° C.+/−0.2, cutoff 60 seconds) as mean+SEM: (a) non-habituated and (b) habituated trial (**, p<0.01, t-test).

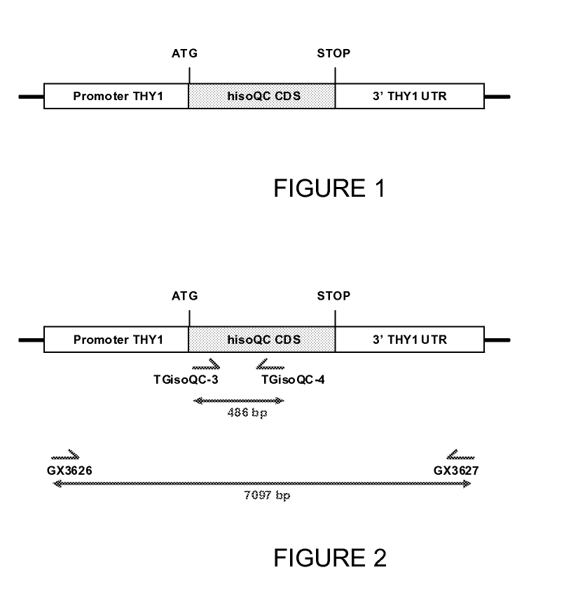

### DETAILED DESCRIPTION OF THE INVENTION

According to one embodiment of the present disclosure, there is provided a transgenic non-human animal for overexpressing isoQC, comprising cells containing a DNA transgene encoding human isoQC, characterized in that said human isoQC comprises the amino acid sequence of SEQ ID NO: 1 or an amino acid sequence having at least 75% sequence identity to the amino acid sequence of SEQ ID NO: 1 or a fragment or derivative of the amino acid sequence of SEQ ID NO: 1.

SEQ ID NO: 1 disclosed herein is also described as “human isoQC Met I, protein” and SEQ ID NO: 11 in WO 2008/034891 and “GenBank Accession Number NM_{—}017659” and SEQ ID NO: 16 in WO 2008/087197.

When amino acids, peptides or polypeptides are referred to herein, it will be appreciated that the amino acid residue will be represented by a one-letter or a three-letter designation, corresponding to the trivial name of the amino acid, in accordance with the following conventional list:

| Amino Acid | One-Letter Symbol | Three-Letter Symbol |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |

In one embodiment, the human isoQC has an amino acid sequence having at least 80% sequence identity to the amino acid sequence of SEQ ID NO: 1, such as a sequence identity selected from any one of 85%, 86%, 87%, 88%, 89%, 90%, 91%, 92%, 93%, 94%, 95%, 96%, 97%, 98% or 99% sequence identity to the amino acid sequence of SEQ ID NO: 1. In a particular embodiment, the human isoQC consists of the amino acid sequence of SEQ ID NO: 1.

In one embodiment, the human isoQC comprises a fragment or derivative of the amino acid sequence of SEQ ID NO: 1. It will be appreciated that when the human isoQC comprises a fragment of the amino acid sequence of SEQ ID NO: 1 it will be required to be a fragment which retains some or all of the function of the full-length isoQC amino acid sequence described in SEQ ID NO: 1. References herein to “derivative of the amino acid sequence of SEQ ID NO: 1” include modifications of the amino acid sequence of SEQ ID NO: 1.

Individual substitutions, deletions or additions, which alter, add or delete a single amino acid or a small percentage of amino acids (typically less than 10%, more typically less than 5%, and still more typically less than 1%.) A “modification” of the amino acid sequence encompasses conservative substitutions of the amino acid sequence. Conservative substitution tables providing functionally similar amino acids are well known in the art. The following six groups each contain amino acids that are conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (1), Leucine (L), Methionine (M), Valine (V); and
6) Phenylalanine (F), Tyrosine (Y), Tryptophan (W).

Other minor modifications are included within the sequence so long as the polypeptide retains some or all of the structural or functional characteristics of the isoQC polypeptide of SEQ ID NO: 1. Exemplary structural or functional characteristics include sequence identity or substantial similarity, antibody reactivity, the presence of conserved structural domains such as RNA binding domains or acidic domains.

It will be appreciated that references herein to isoQC refer to isoglutaminyl peptide cyclotransferase (also known as QPCTL or QC-like enzyme) and that QC (glutaminyl-peptidecyclotransferase (EC 2.3.2.5.)) and isoQC have identical or similar enzyme activity, further defined as QC activity.

The term “QC activity” as used herein is defined as intramolecular cyclization of N-terminal glutamine residues into pyroglutamic acid (pGlu*) or of N-terminal L-homoglutamine or L-R-homoglutamine to a cyclic pyro-homoglutamine derivative under liberation of ammonia. See Schemes 1 and 2.

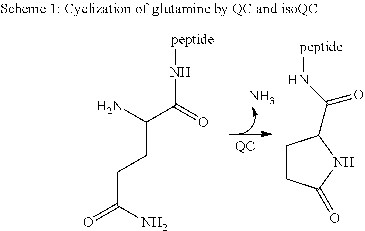

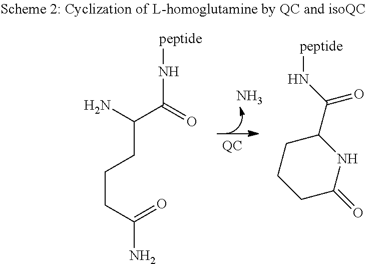

References herein to the term “QC-related disease” or “QC-related disorder refers to all diseases, disorders or conditions that are modulated by QC or isoQC.

References herein to the term “transgene” include a segment of DNA that has been incorporated into a host genome or is capable of autonomous replication in a host cell and is capable of causing the expression of one or more cellular products. Exemplary transgenes will provide the host cell, or animals developed therefrom, with a novel phenotype relative to the corresponding non-transformed cell or animal.

In one embodiment, the DNA transgene comprises the nucleotide sequence of SEQ ID NO: 2 or substantially the same nucleotide sequence of SEQ ID NO: 2.

The isoQC polynucleotides comprising the transgene of the present disclosure include isoQC cDNA and shall also include modified isoQC cDNA. As used herein, a “modification” of a nucleic acid can include one or several nucleotide additions, deletions, or substitutions with respect to a reference sequence. A modification of a nucleic acid can include substitutions that do not change the encoded amino acid sequence due to the degeneracy of the genetic code, or which result in a conservative substitution. Such modifications can correspond to variations that are made deliberately, such as the addition of a Poly A tail, or variations which occur as mutations during nucleic acid replication.

References herein to “substantially the same nucleotide sequence” refers to DNA having sufficient identity to the reference polynucleotide, such that it will hybridize to the reference nucleotide under moderately stringent, or higher stringency, hybridization conditions. DNA having “substantially the same nucleotide sequence” as the reference nucleotide sequence, can have an identity ranging from at least 60% to at least 95% with respect to the reference nucleotide sequence.

The phrase “moderately stringent hybridization” refers to conditions that permit a target-nucleic acid to bind a complementary nucleic acid. The hybridized nucleic acids will generally have an identity within a range of at least about 60% to at least about 95%. Moderately stringent conditions are conditions equivalent to hybridization in 50% formamide, 5× Denhart's solution, 5× saline sodium phosphate EDTA buffer (SSPE), 0.2% SDS (Aldrich) at about 42° C., followed by washing in 0.2×SSPE, 0.2% SDS (Aldrich), at about 42° C. High stringency hybridization refers to conditions that permit hybridization of only those nucleic acid sequences that form stable hybrids in 0.018M NaCl at about 65° C., for example, if a hybrid is not stable in 0.018M NaCl at about 65° C., it will not be stable under high stringency conditions, as contemplated herein. High stringency conditions can be provided, for example, by hybridization in 50% formamide, 5× Denhart's solution, 5×SSPE, 0.2% SDS at about 42° C., followed by washing in 0.1×SSPE, and 0.1% SDS at about 65° C.

Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al. (Current Protocols in Molecular Biology (Supplement 47), John Wiley & Sons, New York (1999)).

In one embodiment, the DNA transgene has a nucleotide sequence having at least 75% sequence identity to the nucleotide sequence of SEQ ID NO: 2, such as a sequence identity selected from any one of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% sequence identity to the nucleotide sequence of SEQ ID NO: 2. In a particular embodiment, the DNA transgene consists of the nucleotide sequence of SEQ ID NO: 2.

SEQ ID NO: 2 disclosed herein is also described as “human isoQC Met I, nucleic acid” and SEQ ID NO: 2 in WO 2008/034891 and SEQ ID NO: 27 in WO 2008/087197.

In one embodiment, the transgene is operably linked to a tissue-specific promoter. References herein to the term “operably linked” include references to a DNA sequence and a regulatory sequence(s) are connected in such a way as to permit gene expression when the appropriate molecules (e.g., transcriptional activator proteins) are bound to the regulatory sequence(s).

The present disclosure further provides a DNA construct comprising the isoQC transgene as described above. As used herein, the term “DNA construct” refers to a specific arrangement of genetic elements in a DNA molecule.

References herein to the term “construct” includes a recombinant nucleic acid, generally recombinant DNA, that has been generated for the purpose of the expression of a specific nucleotide sequence(s), or is to be used in the construction of other recombinant nucleotide sequences. The recombinant nucleic acid can encode e.g. a chimeric or humanized polypeptide.

If desired, the DNA constructs can be engineered to be operatively linked to appropriate expression elements such as promoters or enhancers to allow expression of a genetic element in the DNA construct in an appropriate cell or tissue. The use of the expression control mechanisms allows for the targeted delivery and expression of the gene of interest. For example, the constructs of the present disclosure may be constructed using an expression cassette which includes in the 5′-3′ direction of transcription, a transcriptional and translational initiation region associated with gene expression in brain tissue, DNA encoding a mutant or wild-type isoQC protein, and a transcriptional and translational termination region functional in the host animal. One or more introns also can be present. The transcriptional initiation region can be endogenous to the host animal or foreign or exogenous to the host animal.

The DNA constructs described herein, may be incorporated into vectors for propagation or transfection into appropriate cells to generate isoQC overexpressing mutant non-human mammals and are also comprised by the present disclosure. One skilled in the art can select a vector based on desired properties, for example, for production of a vector in a particular cell such as a mammalian cell or a bacterial cell.

Vectors can contain a regulatory element that provides tissue specific or inducible expression of an operatively linked nucleic acid. One skilled in the art can readily determine an appropriate tissue-specific promoter or enhancer that allows expression of isoQC polypeptides in a desired tissue. It should be noted that tissue-specific expression as described herein does not require a complete absence of expression in tissues other than the preferred tissue. Instead, “cell-specific” or “tissue-specific” expression refers to a majority of the expression of a particular gene of interest in the preferred cell type or tissue.

Any of a variety of inducible promoters or enhancers can also be included in the vector for expression of a isoQC polypeptide or nucleic acid that can be regulated. Such inducible systems, include, for example, tetracycline inducible System (Gossen & Bizard, Proc. Natl. Acad. Sci. USA, 89:5547-5551 (1992); Gossen et al., Science, 268:17664769 (1995); Clontech, Palo Alto, Calif.); metallothionein promoter induced by heavy metals; insect steroid hormone responsive to ecdysone or related steroids such as muristerone (No et al., Proc. Natl. Acad. Sci. USA, 93:3346-3351 (1996); Yao et al., Nature, 366:476-479 (1993); Invitrogen, Carlsbad, Calif.); mouse mammary tumor virus (MMTV) induced by steroids such as glucocorticoid and estrogen (Lee et al., Nature, 294:228-232 (1981); and heat shock promoters inducible by temperature changes; the rat neuron specific enolase gene promoter (Forss-Petter, et al., Neuron 5; 197-197 (1990)); the human β-actin gene promoter (Ray, et al., Genes and Development (1991) 5:2265-2273); the human platelet derived growth factor B (PDGF-B) chain gene promoter (Sasahara, et al., Cell (1991) 64:217-227); the rat sodium channel gene promoter (Maue, et al., Neuron (1990) 4:223-231); the human copper-zinc superoxide dismutase gene promoter (Ceballos-Picot, et al., Brain Res. (1991) 552:198-214); and promoters for members of the mammalian POU-domain regulatory gene family (Xi et al., (1989) Nature 340:35-42).

Regulatory elements, including promoters or enhancers, can be constitutive or regulated, depending upon the nature of the regulation, and can be regulated in a variety of tissues, or one or a few specific tissues. The regulatory sequences or regulatory elements are operatively linked to one of the polynucleotide sequences of the present disclosure such that the physical and functional relationship between the polynucleotide sequence and the regulatory sequence allows transcription of the polynucleotide sequence. Vectors useful for expression in eukaryotic cells can include, for example, regulatory elements including the CAG promoter, the SV40 early promoter, the cytomegalovirus (CMV) promoter, the mouse mammary tumor virus (MMTV) steroid-inducible promoter, Pgtf, Moloney marine leukemia virus (MMLV) promoter, thy-1 promoter and the like.

If desired, the vector can contain a selectable marker. As used herein, a “selectable marker” refers to a genetic element that provides a selectable phenotype to a cell in which the selectable marker has been introduced. A selectable marker is generally a gene whose gene product provides resistance to an agent that inhibits cell growth or kills a cell. A variety of selectable markers can be used in the DNA constructs of the present disclosure, including, for example, Neo, Hyg, hisD, Gpt and Ble genes, as described, for example in Ausubel et al. (Current Protocols in Molecular Biology (Supplement 47), John Wiley & Sons, New York (1999)) and U.S. Pat. No. 5,981,830. Drugs useful for selecting for the presence of a selectable marker include, for example, G418 for Neo, hygromycin for Hyg, histidinol for hisD, xanthine for Gpt, and bleomycin for Ble (see Ausubel et al, supra, (1999); U.S. Pat. No. 5,981,830). DNA constructs of the present disclosure can incorporate a positive selectable marker, a negative selectable marker, or both (see, for example, U.S. Pat. No. 5,981,830).

### Non-Human Transgenic Animals

The present disclosure provides a non-human transgenic animal whose genome comprises a transgene encoding an isoQC polypeptide. References herein to the term “transgenic animal” include a non-human animal, usually a mammal, having a non-endogenous nucleic acid sequence present as an extrachromosomal element in a portion of its cells or stably integrated into its germ line DNA.

In one embodiment, the animal is heterozygous for the transgene. In an alternative embodiment, the animal is homozygous for the transgene. In a further embodiment, the animal is a mouse.

The DNA fragment can be integrated into the genome of a transgenic animal by any method known to those skilled in the art. The DNA molecule containing the desired gene sequence can be introduced into pluripotent cells, such as ES cells, by any method that will permit the introduced molecule to undergo recombination at its regions of homology. Techniques that can be used include, but are not limited to, calcium phosphate/DNA co-precipitates, microinjection of DNA into the nucleus, electroporation, bacterial protoplast fusion with intact cells, transfection, and polycations, (e.g., polybrene, polyornithine, etc.) The DNA can be single or double stranded DNA, linear or circular. (See for example, Hogan et al., Manipulating the Mouse Embryo: A Laboratory Manual Cold Spring Harbor Laboratory (1986); Hogan et al., Manipulating the Mouse Embryo: A Laboratory Manual, second ed., Cold Spring Harbor Laboratory (1994), U.S. Pat. Nos. 5,602,299; 5,175,384; 6,066,778; 4,873,191 and 6,037,521; retrovirus mediated gene transfer into germ lines (Van der Putten et al., Proc. Natl. Acad. Sci. USA 82:6148-6152 (1985)); gene targeting in embryonic stem cells (Thompson et al., Cell 56:313-321 (1989)); electroporation of embryos (Lo, Mol Cell. Biol. 3:1803-1814 (1983)); and sperm-mediated gene transfer (Lavitrano et al., Cell 57:717-723 (1989)).

For example, the zygote is a good target for microinjection, and methods of microinjecting zygotes are well known (see U.S. Pat. No. 4,873,191).

Embryonal cells at various developmental stages can also be used to introduce transgenes for the production of transgenic animals. Different methods are used depending on the stage of development of the embryonal cell. Such transfected embryonic stem (ES) cells can thereafter colonize an embryo following their introduction into the blastocoele of a blastocyst-stage embryo and contribute to the germ line of the resulting chimeric animal (reviewed in Jaenisch, Science 240:1468-1474 (1988)). Prior to the introduction of transfected ES cells into the blastocoele, the transfected ES cells can be subjected to various selection protocols to enrich the proportion of ES cells that have integrated the transgene if the transgene provides a means for such selection. Alternatively, PCR can be used to screen for ES cells that have integrated the transgene.

In addition, retroviral infection can also be used to introduce transgenes into a non-human animal. The developing non-human embryo can be cultured in vitro to the blastocyst stage. During this time, the blastomeres can be targets for retroviral infection (Janenich, Proc. Nati. Acad. Sci. USA 73:1260-1264 (1976)). Efficient infection of the blastomeres is obtained by enzymatic treatment to remove the zona pellucida (Hogan et al., supra, 1986). The viral vector system used to introduce the transgene is typically a replication-defective retrovirus carrying the transgene (Jahner et al., Proc. Natl. Acad Sci. USA 82:6927-6931 (1985); Van der Putten et al., Proc. Natl. Acad Sci. USA 82:6148-6152 (1985)). Transfection is easily and efficiently obtained by culturing the blastomeres on a monolayer of virus-producing cells (Van der Putten, supra, 1985; Stewart et al., EMBO J. 6:383-388 (1987)). Alternatively, infection can be performed at a later stage. Virus or virus-producing cells can be injected into the blastocoele (Jahner D. et al., Nature 298:623-628 (1982)). Most of the founders will be mosaic for the transgene since incorporation occurs only in a subset of cells, which form the transgenic animal. Further, the founder can contain various retroviral insertions of the transgene at different positions in the genome, which generally will segregate in the offspring. In addition, transgenes may be introduced into the germline by intrauterine retroviral infection of the mid-gestation embryo (Jahner et al., supra, 1982). Additional means of using retroviruses or retroviral vectors to create transgenic animals known to those of skill in the art involves the micro-injection of retroviral particles or mitomycin C-treated cells producing retrovirus into the perivitelline space of fertilized eggs or early embryos (WO 90/08832 (1990); Haskell and Bowen, Mal. Reprod. Dev. 40:386 (1995)).

Any other technology to introduce transgenes into a non-human animal, e.g. the knock-in or the rescue technologies can also be used to solve a problem of the present disclosure. The knock-in technology is well known in the art as described e.g. in Casas et al. (2004) Am J Pathol 165, 1289-1300.

Once the founder animals are produced, they can be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic mice to produce mice homozygous for a given integration site in order to both augment expression and eliminate the need for screening of animals by DNA analysis; crossing of separate homozygous lines to produce compound heterozygous or homozygous lines; breeding animals to different inbred genetic backgrounds so as to examine effects of modifying alleles on expression of the transgene and the effects of expression.

The transgenic animals are screened and evaluated to select those animals having the phenotype of interest. Initial screening can be performed using, for example, Southern blot analysis or PCR techniques to analyze animal tissues to verify that integration of the transgene has taken place. The level of mRNA expression of the transgene in the tissues of the transgenic animals can also be assessed using techniques which include, but are not limited to, Northern blot analysis of tissue samples obtained from the animal, in situ hybridization analysis, and reverse transcriptase-PCR (rt-PCR). Samples of the suitable tissues can be evaluated immunocytochemically using antibodies specific for isoQC or with a tag such as EGFP. The transgenic non-human mammals can be further characterized to identify those animals having a phenotype useful in methods of the present disclosure. In particular, transgenic non-human mammals overexpressing isoQC can be screened using the methods disclosed herein. For example, tissue sections can be viewed under a fluorescent microscope for die present of fluorescence, indicating the presence of the reporter gene.

Another method to affect tissue specific expression of the isoQC protein is through the use of tissue-specific promoters. Non-limiting examples of suitable tissue-specific promoters include the albumin promoter (liver-specific; Pinkert et al., (1987) Genes Dev. 1:268-277); lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al., (1983) Cell 33:729-740; Queen and Baltimore (1983) Cell 33:741-748), neuron-specific promoters (e.g., the neurofilament promoter, the Thy-1 promoter or the Bri-protein promoter; Sturchler-Pierrat et al., (1997) Proc. Natl. Acad Sci. USA 94:13287-13292, Byrne and Ruddle (1989) PNAS 86:5473-5477), pancreas-specific promoters (Edlund et al., (1985) Science 230:912-916), cardiac specific expression (alpha myosin heavy chain promoter, Subramaniam, A, Jones W K, Gulick J, Wert S, Neumann J, and Robbins J. Tissue-specific regulation of the alpha-myosin heavy chain gene promoter in transgenic mice. J Biol Chem 266: 24613-24620, 1991.), and mammary gland-specific promoters (e.g., milk whey promoter; U.S. Pat. No. 4,873,316 and European Application Publication No. 264, 166).

The present disclosure further provides an isolated cell containing a DNA construct of the present disclosure. The DNA construct can be introduced into a cell by any of the well-known transfection methods (Sambrook et al., Molecular Cloning: A Laboratory Manual, 2nd ed., Cold Spring Harbor Press, Plainview, N.Y. (1989); Ausubel et al., supra, (1999)). Alternatively, the cell can be obtained by isolating a cell from a mutant non-human mammal created as described herein. Thus, the present disclosure provides a cell isolated from an isoQC mutant non-human mammal of the present disclosure, in particular, an isoQC mutant mouse. The cells can be obtained from a homozygous isoQC mutant non-human mammal such as a mouse or a heterozygous isoQC mutant non-human mammal such as a mouse.

According to a further embodiment of the present disclosure, there is provided a transgenic mouse comprising a transgenic nucleotide sequence encoding isoQC, which comprises the nucleotide sequence of SEQ ID NO: 2 or substantially the same nucleotide sequence of SEQ ID NO: 2, operably linked to a promoter, integrated into the genome of the mouse, wherein the mouse demonstrates a phenotype that can be reversed or ameliorated with an isoQC inhibitor

### Effectors

Effectors, as that term is used herein, are defined as molecules that bind to enzymes and increase (promote) or decrease (inhibit) their activity in vitro or in vivo. Some enzymes have binding sites for molecules that affect their catalytic activity; a stimulator molecule is called an activator. Enzymes may even have multiple sites for recognizing more than one activator or inhibitor. Enzymes can detect concentrations of a variety of molecules and use that information to vary their own activities.

Effectors can modulate enzymatic activity because enzymes can assume both active and inactive conformations: activators are positive effectors, inhibitors are negative effectors. Effectors act not only at the active sites of enzymes, but also at regulatory sites, or allosteric sites, terms used to emphasize that the regulatory site is an element of the enzyme distinct from the catalytic site and to differentiate this form of regulation from competition between substrates and inhibitors at the catalytic site (Darnell, J., Lodish, H. and Baltimore, D. 1990, Molecular Cell Biology 2nd Edition, Scientific American Books, New York, page 63).

### Assays and Identification of Therapeutic Agents

The methods and compositions of the present disclosure are particularly useful in the evaluation of effectors of isoQC, preferably activity decreasing effectors of isoQC, i.e. isoQC inhibitors, and for the development of drugs and therapeutic agents for the treatment and prevention of a disease selected from mild cognitive impairment, Alzheimer's disease, Familial British Dementia, Familial Danish Dementia, neurodegeneration in Down Syndrome, Huntington's disease, Kennedy's disease, ulcer disease, duodenal cancer with or w/o Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, rheumatoid arthritis, atherosclerosis, pancreatitis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

The transgenic animal or the cells of the transgenic animal of the present disclosure can be used in a variety of screening assays. Thus, according to a further embodiment of the present disclosure, there is provided a method of screening for biologically active agents that inhibit or promote isoQC production in vivo, comprising:
(a) administering a test agent to the transgenic non-human animal as defined herein;
and
(b) determining the effect of the agent on the amount of isoQC produced.

According to a yet further embodiment of the present disclosure there is provided a method of screening for therapeutic agents that inhibit or promote isoQC activity comprising:
(a) administering test agents to the transgenic mouse as defined herein;
(b) evaluating the effects of the test agent on the neurological phenotype of the mouse; and
(c) selecting a test agent which inhibits or promotes isoQC activity.

For example, any of a variety of potential agents suspected of affecting isoQC and amyloid accumulation, as well as the appropriate antagonists and blocking therapeutic agents, can be screened by administration to the transgenic animal and assessing the effect of these agents upon the function and phenotype of the cells and on the (neurological) phenotype of the transgenic animals.

Behavioral studies may also be used to test potential therapeutic agents, such as those studies designed to assess motor skills, learning and memory deficits. An example of such a test is the Morris Water maze (Morris (1981) Learn Motivat 12:239-260). Additionally, behavioral studies may include evaluations of locomotor activity such as with the rotor-rod and the open field.

The methods of the present disclosure can advantageously use cells isolated from a homozygous or heterozygous isoQC mutant non-human mammal, to study amyloid accumulation as well as to test potential therapeutic compounds. The methods of the present disclosure can also be used with cells expressing isoQC such as a transfected cell line.

According to a further embodiment of the present disclosure, there is provided a cell or cell line derived from the transgenic non-human animal as defined herein.

A cell overexpressing isoQC can be used in an in vitro method to screen compounds as potential therapeutic agents for treating a isoQC-related disease. In such a method, a compound is contacted with a cell overexpressing isoQC, a transfected cell or a cell derived from an isoQC mutant non-human animal, and screened for alterations in a phenotype associated with expression of isoQC. The changes in Aβ production in the cellular assay and the transgenic animal can be assessed by methods well known to those skilled in the art.

An isoQC fusion polypeptide such as isoQC can be particularly useful for such screening methods since the expression of isoQC can be monitored by fluorescence intensity. Other exemplary fusion polypeptides include other fluorescent proteins, or modifications thereof, glutathione S transferase (GST), maltose binding protein, poly His, and the like, or any type of epitope tag. Such fusion polypeptides can be detected, for example, using antibodies specific to the fusion polypeptides. The fusion polypeptides can be an entire polypeptide or a functional portion thereof so long as the functional portion retains desired properties, for example, antibody binding activity or fluorescence activity.

The present disclosure further provides a method of identifying a potential therapeutic agent for use in treating the diseases as mentioned above. The method includes the steps of contacting a cell containing a DNA construct comprising polynucleotides encoding an isoQC polypeptide with a compound and screening the cell for decreased isoQC production, thereby identifying a potential therapeutic agent for use in treating isoQC-related diseases. The cell can be isolated from a transgenic non-human mammal having nucleated cells containing the isoQC DNA construct. Alternatively, the cell can contain a DNA construct comprising a nucleic acid encoding a green fluorescent protein fusion, or other fusion polypeptide, with an isoQC polypeptide.

Additionally, cells expressing an isoQC polypeptide can be used in a preliminary screen to identify compounds as potential therapeutic agents having activity that alters a phenotype associated with isoQC expression. As with in vivo screens using isoQC mutant non-human mammals, an appropriate control cell can be used to compare the results of the screen. The effectiveness of compounds identified by an initial in vitro screen using cells expressing isoQC can be further tested in vivo using the isoQC mutant non-human mammals of the present disclosure, if desired. Thus, the present disclosure provides methods of screening a large number of compounds using a cell-based assay, for example, using high throughput screening, as well as methods of further testing compounds as therapeutic agents in an animal model of Aβ-related disorders.

The non-human transgenic animals whose genome comprises a transgene encoding an isoQC polypeptide can be used to investigate the physiological function of isoQC in vivo.

In one embodiment, the isoQC transgenic animals of the present disclosure are crossbred with existing animal models, that are acknowledged disease specific animal models. Such crossbred animals can be used to determine the effect of overexpressed recombinant isoQC or increased isoQC activity on the outbreak, course and severity of said specific diseases.

A suitable method comprises the following steps:
(a) Crossbreeding of the isoQC transgenic non-human animals of the present disclosure with a non-human animal model, which is specific for a desired disease,
(b) Breeding and ageing the crossbred animals and the disease specific animals;
(c) Monitoring the disease state age-dependently in the crossbred animals,
(d) As a control group, monitoring the disease state age-dependently in the disease specific animal models that are not transgenic for isoQC,
(e) Calculating the differences in the disease state in the crossbred animals versus the disease specific animals, and
(f) Determining the effect of the isoQC transgene on the disease state.

Furthermore, said crossbred animals are suitable for use in methods of screening for activity decreasing effectors of isoQC (isoQC inhibitors). A suitable screening method comprises:
(a) Crossbreeding of the isoQC transgenic non-human animals of the present disclosure with a non-human animal model, which is specific for a desired disease,
(b) Administering a test agent to a treatment group of crossbred animals,
(c) Administering a placebo to a control group of crossbred animals,
(d) Monitoring the disease state age-dependently in the crossbred animals,
(e) Monitoring the disease state age-dependently in the control group,
(f) Calculating the differences in the disease state in the treatment group versus the control group, and
(g) Determining the effect of the test agent on the disease state.

Suitably, the crossbred animals are heterozygous for the isoQC transgene. More preferably, the crossbred animals are homozygous for the isoQC transgene.

The recombinant isoQC, which is overexpressed in the aforementioned crossbred non-human animals, suitably leads to one or more of the following effects on the disease state: an earlier outbreak of the specific disease, an accelerated course of the specific disease or a more severe course of the specific disease.

Another effect of the overexpressed isoQC could be the increase or decrease of the level of one or more isoQC substrates in the crossbred non-human animals.

A particular preferred embodiment is the use of this method for screening of isoQC inhibitors.

Suitably, this method is used for the screening of isoQC inhibitors for the treatment of a disease selected from mild cognitive impairment, Alzheimer's disease, Familial British Dementia, Familial Danish Dementia, neurodegeneration in Down Syndrome, Huntington's disease, Kennedy's disease, ulcer disease, duodenal cancer with or w/o Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, rheumatoid arthritis, atherosclerosis, pancreatitis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

In a further preferred embodiment, this method is used for the screening of isoQC inhibitors for the treatment of Alzheimer's disease or neurodegeneration in Down syndrome.

In yet another preferred embodiment, this method is used for the screening of isoQC inhibitors for the treatment of Familial British Dementia or Familial Danish Dementia.

Furthermore, this method is preferably used for the screening of isoQC inhibitors for the treatment of a disease selected from rheumatoid arthritis, atherosclerosis, restenosis, and pancreatitis.

The efficacy of isoQC inhibitors for the treatment of Alzheimer's Disease, Familial British Dementia or Familial Danish Dementia and, e.g. neurodegeneration in Down Syndrome can be tested in existing animal models of Alzheimer's disease.

isoQC may be involved in the formation of pyroglutamic acid that favors the aggregation of amyloid β-peptides. Therefore, a suitable isoQC substrate, which can be monitored when the above methods are employed, is one selected from [Glu3]Aβ3-40/42/43 or [Glu11]Aβ11-40/42/43. These peptides are involved in the onset and progression of Alzheimer's disease and neurodegeneration in Down Syndrome. Recombinant isoQC, which is expressed in the crossbred non-human animals of the present disclosure, may lead to one or more of the following effects: earlier formation of at least one of [pGlu3]Aβ3-40/42/43 or [pGlu11]Aβ11-40/42/43, faster formation of at least one of [pGlu3]Aβ3-40/42/43 or [pGlu11]Aβ11-40/42/43 or increased level of at least one of [pGlu3]Aβ3-40/42/43 or [pGlu11]Aβ11-40/42/43.

The isoQC inhibitor, which is selected by employing the screening method in the crossbred non-human animals accordingly leads to the prevention of the formation of at least one of [pGlu3]Aβ3-40/42/43 or [pGlu11]Aβ3-40/42/43 and may subsequently lead to the prevention of the precipitation of amyloid β-peptides and formation of plaques. Finally, said isoQC inhibitor should suitably lead to one or more of the following effects: postponing the outbreak, slowing down the course or reducing the severity of Alzheimer's disease and neurodegeneration in Down Syndrome in the crossbred non-human animals.

Suitable animal models of Alzheimer's Disease are reviewed in McGowan et al., TRENDS in Genetics, Vol. 22, No. May 2006, pp 281-289, and are selected from PDAPP, Tg2576, APP23, TgCRND8, PSEN1M146V or PSEN1M146L, PSAPP, APPDutch, BRI-Aβ40 and BRI-Aβ42, JNPL3, TauP301S, TauV337M, TauR406W, rTg4510, Htau, TAPP, 3×TgAD, as described below. Another suitable model of Alzheimer's disease is the 5XFAD model (Oakley H., et al., Intraneuronal beta-amyloid aggregates, neurodegeneration, and neuron loss in transgenic mice with five familial Alzheimer's disease mutations: potential factors in amyloid plaque formation. J Neurosci. 2006 Oct. 4; 26(40):10129-40).

PDAPP: First mutant APP transgenic model with robust plaque pathology. Mice express a human APP cDNA with the Indiana mutation (APPV717F). Plaque pathology begins between 6-9 months in hemizygous PDAPP mice. There is synapse loss but no overt cell loss and no NFT pathology is observed. This model has been used widely in vaccination therapy strategies.

Tg2576: Mice express mutant APPSWE under control of the hamster prion promoter. Plaque pathology is observed from 9 months of age. These mice have cognitive deficits but no cell loss or NFT pathology. It is one of the most widely used transgenic models.

APP23: Mice express mutant APPSWE under control of the Thy1 promoter. Prominent cerebrovascular amyloid, amyloid deposits are observed from 6 months of age and some hippocampal neuronal loss is associated with amyloid plaque formation.

TgCRND8: Mice express multiple APP mutations (Swedish plus Indiana). Cognitive deficits coincide with rapid extracellular plaque development at ˜3 months of age. The cognitive deficits can be reversed by Aβ vaccination therapy.

PSEN1M146V or PSEN1M146L (lines 6.2 and 8.9, respectively): These models were the first demonstration in vivo that mutant PSEN1 selectively elevates Aβ42. No overt plaque pathology is observed.

PSAPP (Tg2576×PSEN1M146L, PSEN1-A246E+APPSWE): Bigenic transgenic mice, addition of the mutant PSEN1 transgene markedly accelerated amyloid pathology compared with singly transgenic mutant APP mice, demonstrating that the PSEN1-driven elevation of Aβ42 enhances plaque pathology.

APPDutch: Mice express APP with the Dutch mutation that causes hereditary cerebral hemorrhage with amyloidosis-Dutch type in humans. APPDutch mice develop severe congophilic amyloid angiopathy. The addition of a mutant PSEN1 transgene redistributes the amyloid pathology to the parenchyma indicating differing roles for Aβ40 and Aβ42 in vascular and parenchymal amyloid pathology.

BRI-Aβ40 and BRI-Aβ42: Mice express individual Aβ isoforms without APP over-expression. Only mice expressing Aβ42 develop senile plaques and CAA, whereas BRI-Aβ40 mice do not develop plaques, suggesting that Aβ42 is essential for plaque formation.

JNPL3: Mice express 4R0N MAPT with the P301 L mutation. This is the first transgenic model, with marked tangle pathology and cell loss, demonstrating that MAPT alone can cause cellular damage and loss. JNPL3 mice develop motor impairments with age owing to servere pathology and motor neuron loss in the spinal cord.

TauP301S: Tansgenic mice expressing the shortest isoform of 4R MAPT with the P301S mutation. Homozygous mice develop severe paraparesis at 5-6 months of age with widespread neurofibrillary pathology in the brain and spinal cord and neuronal loss in the spinal cord.

TauV337M: Low level synthesis of 4R MAPT with the V337M mutation (1/10 endogenous MAPT) driven by the promoter of platelet-derived growth factor (PDGF). The development of neurofibrillary pathology in these mice suggests the nature of the MAPT rather than absolute MAPT intracellular concentration drives pathology.

TauR406W: Mice expressing 4R human MAPT with the R406W mutation under control of the CAMKII promoter. Mice develop MAPT inclusions in the forebrain from 18 months of age and have impaired associative memory.

rTg4510: Inducible MAPT transgenic mice using the TET-off system. Abnormal MAPT pathology occurs from one month of age. Mice have progressive NFT pathology and severe cell loss. Cognitive deficits are evident from 2.5 months of age. Turning off the transgene improves cognitive performance but NT pathology worsens.

Htau: Transgenic mice expressing human genomic MAPT only (mouse MAPT knocked-out). Htau mice accumulate hyperphosphorylated MAPT from 6 months and develop Thio-S-positive NFT by the time they are 15 months old.

TAPP (Tg2576×JNPL3): Increased MAPT forebrain pathology in TAPP mice compared with JNPL3 suggesting mutant APP or Aβ can affect downstream MAPT pathology.

3×TgAD: Triple transgenic model expressing mutant APPSWE, MAPTP301 L on a PSEN1M146V ‘knock-in’ background (PSNE1-KI). Mice develop plaques from 6 months and MAPT pathology from the time they are 12 months old, strengthening the hypothesis that APP or Aβ can directly influence neurofibrillary pathology.

5XFAD: Mutations in the genes for amyloid precursor protein (APP) and presenilins (PS1, PS2) increase production of beta-amyloid 42 (Abeta42) and cause familial Alzheimer's disease (FAD). Transgenic mice that express FAD mutant APP and PS1 overproduce Abeta42 and exhibit amyloid plaque pathology similar to that found in AD, but most transgenic models develop plaques slowly. To accelerate plaque development and investigate the effects of very high cerebral Abeta42 levels, APP/PS1 double transgenic mice were generated that coexpress five FAD mutations (5XFAD mice) and additively increase Abeta42 production. 5XFAD mice generate Abeta42 almost exclusively and rapidly accumulate massive cerebral Abeta42 levels. Amyloid deposition (and gliosis) begins at 2 months and reaches a very large burden, especially in subiculum and deep cortical layers. Intraneuronal Abeta42 accumulates in 5XFAD brain starting at 1.5 months of age (before plaques form), is aggregated (as determined by thioflavin S staining), and occurs within neuron soma and neurites. Some amyloid deposits originate within morphologically abnormal neuron soma that contain intraneuronal Abeta. Synaptic markers synaptophysin, syntaxin, and postsynaptic density-95 decrease with age in 5XFAD brain, and large pyramidal neurons in cortical layer 5 and subiculum are lost. In addition, levels of the activation subunit of cyclin-dependent kinase 5, p25, are elevated significantly at 9 months in 5XFAD brain. Finally, 5XFAD mice have impaired memory in the Y-maze.

Suitable study designs are conventional. isoQC inhibitors could be applied via the drinking solution or chow, or any other conventional route of administration, e.g. orally, intravenously or subcutaneously.

In regard to Alzheimer's disease and neurodegeneration in Down syndrome, the efficacy of the isoQC inhibitors can be assayed by sequential extraction of Aβ using SDS and formic acid. Initially, the SDS and formic acid fractions containing the highest Aβ concentrations can be analyzed using an ELISA quantifying total Aβ(x-42) or Aβ(x-40) as well as [pGlu3]Aβ3-40/42/43 or [pGlu11]Aβ11-40/42/43. In particular, suitable isoQC inhibitors are capable to reduce the formation of [pGlu3]Aβ3-40 or [pGlu3]Aβ3-42. Even preferred are isoQC inhibitors that are capable to reduce the formation of [pGlu11]Aβ11-40 or [pGlu11]Aβ11-42.

An ELISA kit for the quantification of [pGlu3]Aβ3-42 is commercially available from IBL, Cat-no. JP27716.

An ELISA for the quantification of [pGlu3]Aβ3-40 is described by Schilling et al., 2008 (Schilling S, Appl T, Hoffmann T, Cynis H, Schulz K, Jagla W, Friedrich D, Wermann M, Buchholz M, Heiser U, von Hörsten S, Demuth H U. Inhibition of glutaminyl cyclase prevents pGlu-Abeta formation after intracortical/hippocampal microinjection in vivo/in situ. J Neurochem. 2008 August; 106(3): 1225-36.)

Subsequently after isoQC inhibitor treatment, the crossbred non-human animals can be tested regarding behavioral changes. Suitable behavioral test paradigms are, e.g. those, which address different aspects of hippocampus-dependent learning. Examples of such neurological tests are the Morris water maze test and the Fear Conditioning test looking at contextual memory changes (Comery, T A et al, (2005), J Neurosci 25:8898-8902; Jacobsen J S et al, (2006), Proc Natl. Acad. Sci USA 103:5161-5166). Further suitable behavioral tests are outlined in the working examples of the present application. Suitably, the isoQC inhibitors, which are selected by employing the screening methods of the present disclosure, reduce the behavioral changes, or more suitably improve the behavior of the crossbred non-human animals.

The animal model of inflammatory diseases, e.g. atherosclerosis contemplated by the present disclosure can be an existing atherosclerosis animal model, e.g., the apoE deficient mouse. The apolipoprotein E knockout mouse model has become one of the primary models for atherosclerosis (Arterioscler Thromh Vase Biol., 24: 1006-1014, 2004; Trends Cardiovasc Med, 14: 187-190, 2004). The studies with the crossbred non-human animals of the present disclosure may be performed as described by Johnson et al. in Circulation, 111: 1422-1430, 2005, or using modifications thereof. Apolipoprotein E-Deficient Mouse Model Apolipoprotein E (apoE) is a component of several plasma lipoproteins, including chylomicrons, VLDL, and HDL. Receptor-mediated catabolism of these lipoprotein particles is mediated through the interaction of apoE with the LDL receptor (LDLR) or with LDLR-related protein (LRP). ApoE-deficient mice exhibit hypercholesterolemia and develop complex atheromatous lesions similar to those seen in humans. The efficacy of the compounds of the present disclosure was also evaluated using this animal model.

Other animal models for inflammatory diseases, which are suitable for use in the aforementioned screening method, include those where inflammation is initiated by use of an artificial stimulus. Such animal models are the thioglycollate-induced inflammation model, the collagen-induced arthritis model, the antibody induced arthritis model and models of restenosis (e.g. the effects of the test compounds on rat carotid artery responses to the balloon catheter injury). Such artificial stimuli can be used to initiate an inflammatory response in the crossbred non-human animal models of the present disclosure.

In inflammatory diseases, chemotactic cytokines play a role. Chemotactic cytokines (chemokines) are proteins that attract and activate leukocytes and are thought to play a fundamental role in inflammation. Chemokines are divided into four groups categorized by the appearance of N-terminal cysteine residues (“C”-; “CC”-; “CXC”- and “CX3C”-chemokines). “CXC”-chemokines preferentially act on neutrophils. In contrast, “CC”-chemokines attract preferentially monocytes to sites of inflammation. Monocyte infiltration is considered to be a key event in a number of disease conditions (Gerard, C. and Rollins, B. J. (2001) Nat. Immunol 2, 108-115; Bhatia, M., et al., (2005) Pancreatology. 5, 132-144; Kitamoto, S., Egashira, K., and Takeshita, A. (2003) J Pharmacol Sci. 91, 192-196). The MCP family, as one family of chemokines, consists of four members (MCP-1-4), displaying a preference for attracting monocytes but showing differences in their potential (Luini, W., et al., (1994) Cytokine 6, 28-31; Uguccioni, M., et al., (1995) Eur J Immunol 25, 64-68). The chemokines CCL2 (MCP-1), CCL8 (MCP-2), CCL7 (MCP-3), CCL13 (MCP-1), CCL16, CCL18 bear a glutamine (Gin) residue at the N-terminus and are therefore substrates of isoQC.

Accordingly, isoQC may be involved in the formation of pyroglutamic acid at the N-terminus of the chemokines CCL2, CCL8, CCL7, CCL13, CCL 16, and CCL 18 that stabilizes these chemokines against degradation by proteases and aminopeptidases and thereby maintains their biological activity in chemotaxis. Recombinant isoQC, which is expressed in the crossbred non-human animals of the present disclosure, may lead to one or more of the following effects: earlier formation of at least one of [pGlu1]CCL2, [pGlu1]CCL8, [pGlu1]CCL7, [pGlu1]CCL13, [pGlu1]CCL 16, or [pGlu1]CCL 18, faster formation of at least one of [pGlu1]CCL2, [pGlu1]CCL8, [pGlu1]CCL7, [pGlu1]CCL13, [pGlu1]CCL 16, or [pGlu1]CCL 18 or increased level of at least one of [pGlu1]CCL2, [pGlu1]CCL8, [pGlu1]CCL7, [pGlu1]CCL13, [pGlu1]CCL 16, or [pGlu1]CCL 18.

The isoQC inhibitor, which is selected by employing the screening method in the crossbred non-human animals accordingly leads to the prevention of the formation of at least one of [pGlu1]CCL2, [pGlu1]CCL8, [pGlu1]CCL7, [pGlu1]CCL13, [pGlu1]CCL 16, or [pGlu1]CCL 18.

The efficacy of the isoQC inhibitors can be assayed by measuring the inhibition of the chemotaxis of a monocytic cells induced by MCP-1 in vitro and in vivo or by measuring the inflammatory response caused by thioglycollate, collagen, antibody or LPS induction. Effective isoQC inhibitors should show a reduced monocyte infiltration after thioglycollate, collagen, antibody or LPS induction of inflammation.

Furthermore, the inhibition of the formation of [pGlu1]CCL2, [pGlu1]CCL8, [pGlu1]CCL7, [pGlu1]CCL13, [pGlu1]CCL 16, or [pGlu1]CCL 18 can be tested in vitro and in vivo.

In one embodiment, the present disclosure provides the use of activity-decreasing effectors of isoQC, as selected with use of the present inventive animal model, for the suppression of pGlu-Amyloid peptide formation in Mild Cognitive Impairment, Alzheimer's disease, Down Sydrome, Famlilial Danish Dementia and Familial British Dementia.

In a further embodiment, the present disclosure provides the use of activity-increasing effectors of isoQC, as selected with use of the present inventive animal model, for the stimulation of gastrointestinal tract cell proliferation, especially gastric mucosal cell proliferation, epithelial cell proliferation, the differentiation of acid-producing parietal cells and histamine-secreting enterochromaffin-like (ECL) cells, and the expression of genes associated with histamine synthesis and storage in ECL cells, as well as for the stimulation of acute acid secretion in mammals by maintaining or increasing the concentration of active[pGlu^{l}]-Gastrin.

In a further embodiment, the present disclosure provides the use of activity decreasing effectors of isoQC, as selected with use of the present inventive animal model, for the treatment of duodenal ulcer disease and gastric cancer with or without Helicobacter pylori in mammals by decreasing the conversion rate of inactive [Gln¹]Gastrin to active [pGlu^{l}]Gastrin.

In another embodiment, the present disclosure provides the use of activity increasing effectors of isoQC, as selected with use of the present inventive animal model, for the preparation of antipsychotic drugs or for the treatment of schizophrenia in mammals. The effectors of isoQC either maintain or increase the concentration of active [pGlu^{l}]neurotensin.

In a further embodiment, the present disclosure provides the use of activity-lowering effectors of isoQC, as selected with the present inventive animal model, for the preparation of fertilization prohibitive drugs or to reduce the fertility in mammals. The activity lowering effectors of isoQC decrease the concentration of active [pGlu¹]FPP, leading to a prevention of sperm capacitation and deactivation of sperm cells. In contrast it could be shown that activity-increasing effectors of isoQC are able to stimulate fertility in males and to treat infertility.

In another embodiment, the present disclosure provides the use of effectors of isoQC, as selected with use of the present inventive animal model, for the preparation of a medicament for the treatment of pathophysiological conditions, such as suppression of proliferation of myeloid progenitor cells, neoplasia, inflammatory host responses, cancer, malign metastasis, melanoma, psoriasis, rheumatoid arthritis, atherosclerosis, lung fibrosis, liver fibrosis, renal fibrosis, graft rejection, acquired immune deficiency syndrome, impaired humoral and cell-mediated immunity responses, leukocyte adhesion and migration processes at the endothelium.

In a further embodiment, the present disclosure provides the use of effectors of isoQC, as selected with use of the present inventive animal model, for the preparation of a medicament for the treatment of impaired food intake and sleep-wakefulness, impaired homeostatic regulation of energy metabolism, impaired autonomic function, impaired hormonal balance and impaired regulation of body fluids.

In a further embodiment, the present disclosure therefore provides the use of effectors of isoQC, as selected with the present inventive animal model, for the preparation of a medicament for the treatment of Parkinson disease and Huntington's disease.

In another embodiment, the present disclosure provides a general way to reduce or inhibit the enzymatic activity of isoQC by using the test agent selected above.

The agents selected by the above-described screening methods can work by decreasing the conversion of at least one substrate of isoQC (negative effectors, inhibitors), or by increasing the conversion of at least one substrate of isoQC (positive effectors, activators).

According to a further embodiment of the present disclosure, there is provided a method of the treatment or prevention of a isoQC-related disease comprising:
(a) administering the selected test agent as defined herein; and
(b) monitoring the patient for a decreased clinical index for isoQC-related diseases.

In one embodiment, the isoQC-related disease is Alzheimer's disease.

According to a further embodiment of the present disclosure, there is provided a test agent as defined herein for use in the treatment or prevention of a isoQC-related disease, such as Alzheimer's disease.

The compounds of the present disclosure can be converted into acid addition salts, especially pharmaceutically acceptable acid addition salts.

The salts of the compounds of the present disclosure may be in the form of inorganic or organic salts.

The compounds of the present disclosure can be converted into and used as acid addition salts, especially pharmaceutically acceptable acid addition salts. The pharmaceutically acceptable salt generally takes a form in which a basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. All pharmaceutically acceptable acid addition salt forms of the compounds of the present disclosure are intended to be embraced by the scope of this disclosure.

In view of the close relationship between the free compounds and the compounds in the form of their salts, whenever a compound is referred to in this context, a corresponding salt is also intended, provided such is possible or appropriate under the circumstances.

Where the compounds according to this disclosure have at least one chiral center, they may accordingly exist as enantiomers. Where the compounds possess two or more chiral centers, they may additionally exist as diastereomers. It is to be understood that all such isomers and mixtures thereof are encompassed within the scope of the present disclosure. Furthermore, some of the crystalline forms of the compounds may exist as polymorphs and as such are intended to be included in the present disclosure. In addition, some of the compounds may form solvates with water (i.e. hydrates) or common organic solvents, and such solvates are also intended to be encompassed within the scope of this disclosure.

The compounds, including their salts, can also be obtained in the form of their hydrates, or include other solvents used for their crystallization.

In a further embodiment, the present disclosure provides a method of preventing or treating a condition mediated by modulation of the isoQC enzyme activity in a subject in need thereof which comprises administering any of the compounds of the present disclosure or pharmaceutical compositions thereof in a quantity and dosing regimen therapeutically effective to treat the condition. Additionally, the present disclosure includes the use of the compounds of this disclosure, and their corresponding pharmaceutically acceptable acid addition salt forms, for the preparation of a medicament for the prevention or treatment of a condition mediated by modulation of the isoQC activity in a subject. The compound may be administered to a patient by any conventional route of administration, including, but not limited to, intravenous, oral, subcutaneous, intramuscular, intradermal, parenteral and combinations thereof.

In a further preferred form of implementation, the present disclosure relates to pharmaceutical compositions, that is to say, medicaments, that contain at least one compound or test agent as defined herein or salts thereof, optionally in combination with one or more pharmaceutically acceptable carriers or solvents.

The pharmaceutical compositions may, for example, be in the form of parenteral or enteral formulations and contain appropriate carriers, or they may be in the form of oral formulations that may contain appropriate carriers suitable for oral administration. Preferably, they are in the form of oral formulations.

The effectors of isoQC activity administered according to the present disclosure may be employed in pharmaceutically administrable formulations or formulation complexes as inhibitors or in combination with inhibitors, substrates, pseudosubstrates, inhibitors of isoQC expression, binding proteins or antibodies of those enzyme proteins that reduce the isoQC protein concentration in mammals. The compounds of the present disclosure make it possible to adjust treatment individually to patients and diseases, it being possible, in particular, to avoid individual intolerances, allergies and side-effects.

The compounds also exhibit differing degrees of activity as a function of time. The physician providing treatment is thereby given the opportunity to respond differently to the individual situation of patients: he is able to adjust precisely, on the one hand, the speed of the onset of action and, on the other hand, the duration of action and especially the intensity of action.

A preferred treatment method according to the invention represents a new approach for the prevention or treatment of a condition mediated by modulation of the isoQC enzyme activity in mammals. It is advantageously simple, susceptible of commercial application and suitable for use, especially in the treatment of diseases that are based on unbalanced concentration of physiological active isoQC substrates in mammals and especially in human medicine.

The compounds may be advantageously administered, for example, in the form of pharmaceutical preparations that contain the active ingredient in combination with customary additives like diluents, excipients or carriers known from the prior art. For example, they can be administered parenterally (for example i.v. in physiological saline solution) or enterally (for example orally, formulated with customary carriers).

Depending on their endogenous stability and their bioavailability, one or more doses of the compounds can be given per day in order to achieve the desired normalisation of the blood glucose values. For example, such a dosage range in humans may be in the range of from about 0.01 mg to 250.0 mg per day, preferably in the range of about 0.01 to 100 mg of compound per kilogram of body weight.

By administering effectors of isoQC activity to a mammal it could be possible to prevent or alleviate or treat isoQC-related conditions selected from Mild Cognitive Impairment, Alzheimer's disease, Down Syndrome, Familial Danish Dementia, Familial British Dementia, Huntington's Disease, ulcer disease and gastric cancer with or w/o Helicobacter pylori infections, pathogenic psychotic conditions, schizophrenia, infertility, neoplasia, inflammatory host responses, cancer, psoriasis, rheumatoid arthritis, atherosclerosis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, sleep-wakefulness, impaired homeostatic regulation of energy metabolism, impaired autonomic function, impaired hormonal balance and impaired regulation of body fluids.

Further, by administering effectors of isoQC activity to a mammal it could be possible to stimulate gastrointestinal tract cell proliferation, preferably proliferation of gastric mucosal cells, epithelial cells, acute acid secretion and the differentiation of acid producing parietal cells and histamine-secreting enterochromaffin-like cells.

In addition, administration of isoQC inhibitors to mammals may lead to a loss of sperm cell function thus suppressing male fertility. Thus, the prevent invention provides a method for the regulation and control of male fertility and the use of activity lowering effectors of isoQC for the preparation of contraceptive medicaments for males.

Furthermore, by administering effectors of isoQC activity to a mammal it may be possible to suppress the proliferation of myeloid progenitor cells.

The compounds used according to the invention can accordingly be converted in a manner known per se into conventional formulations, such as, for example, tablets, capsules, dragées, pills, suppositories, granules, aerosols, syrups, liquid, solid and cream-like emulsions and suspensions and solutions, using inert, non-toxic, pharmaceutically suitable carriers and additives or solvents. In each of those formulations, the therapeutically effective compounds are preferably present in a concentration of approximately from 0.1 to 80% by weight, more preferably from 1 to 50% by weight, of the total mixture, that is to say, in amounts sufficient for the mentioned dosage latitude to be obtained.

The substances can be used as medicaments in the form of dragées, capsules, bitable capsules, tablets, drops, syrups or also as suppositories or as nasal sprays.

The formulations may be advantageously prepared, for example, by extending the active ingredient with solvents or carriers, optionally with the use of emulsifiers or dispersants, it being possible, for example, in the case where water is used as diluent, for organic solvents to be optionally used as auxiliary solvents.

Examples of excipients useful in connection with the present invention include: water, non-toxic organic solvents, such as paraffins (for example natural oil fractions), vegetable oils (for example rapeseed oil, groundnut oil, sesame oil), alcohols (for example ethyl alcohol, glycerol), glycols (for example propylene glycol, polyethylene glycol); solid carriers, such as, for example, natural powdered minerals (for example highly dispersed silica, silicates), sugars (for example raw sugar, lactose and dextrose); emulsifiers, such as non-ionic and anionic emulsifiers (for example polyoxyethylene fatty acid esters, polyoxyethylene fatty alcohol ethers, alkylsulphonates and arylsulphonates), dispersants (for example lignin, sulphite liquors, methylcellulose, starch and polyvinylpyrrolidone) and lubricants (for example magnesium stearate, talcum, stearic acid and sodium lauryl sulphate) and optionally flavourings.

Administration may be carried out in the usual manner, preferably enterally or parenterally, especially orally. In the case of enteral administration, tablets may contain in addition to the mentioned carriers further additives such as sodium citrate, calcium carbonate and calcium phosphate, together with various additives, such as starch, preferably potato starch, gelatin and the like. Furthermore, lubricants, such as magnesium stearate, sodium lauryl sulphate and talcum, can be used concomitantly for tabletting. In the case of aqueous suspensions or elixirs intended for oral administration, various taste correctives or colourings can be added to the active ingredients in addition to the above-mentioned excipients.

In the case of parenteral administration, solutions of the active ingredients using suitable liquid carriers can be employed. In general, it has been found advantageous to administer, in the case of intravenous administration, amounts of approximately from 0.01 to 2.0 mg/kg, preferably approximately from 0.01 to 1.0 mg/kg, of body weight per day to obtain effective results and, in the case of enteral administration, the dosage is approximately from 0.01 to 2 mg/kg, preferably approximately from 0.01 to 1 mg/kg, of body weight per day.

It may nevertheless be necessary in some cases to deviate from the stated amounts, depending upon the body weight of the experimental animal or the patient or upon the type of administration route, but also on the basis of the species of animal and its individual response to the medicament or the interval at which administration is carried out. Accordingly, it may be sufficient in some cases to use less than the above-mentioned minimum amount, while, in other cases, the mentioned upper limit will have to be exceeded. In cases where relatively large amounts are being administered, it may be advisable to divide those amounts into several single doses over the day. For administration in human medicine, the same dosage latitude is provided. The above remarks apply analogously in that case.

For examples of pharmaceutical formulations, specific reference is made to the examples of WO 2004/098625, pages 50-52, which are incorporated herein by reference in their entirety.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art. Although specific terms have been employed herein, such terms are intended in a descriptive sense and not for purposes of limitation.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

### Production of Transgenic Mice

### Transgenic Mice

An isoenzyme of human glutaminyl cyclase was identified in the human hepatocellular carcinoma cell line Hep-G2. The cDNA was isolated applying standard molecular biology techniques and was subcloned into vector pPCRScript (Stratagene). The correct sequence was verified by DNA sequencing. Afterwards, the respective cDNA was inserted into vector pUC18 containing the murine Thy-1 sequence applying standard molecular biology techniques. All constructs were verified by sequencing. The transgenic mice were generated by male pronuclear injection (PNI) of fertilized C57Bl6 oocytes (PNI, generated by JSW, Graz, Austria). The injected oocytes were then implanted into foster mothers for full term development. The resulting offspring (5 founders) were further characterized for transgene integration by PCR analysis and after crossing with C57Bl6 wildtype mice, the resulting F1 generation was used for the analysis of transgene expression by RT-PCR (n=3-5 each line). Three lines with low, medium and high levels of expression respectively, were selected for further breeding and cross-breeding experiments (tgisoQC-13, tgisoQC-20 and tgisoQC-31).

### PCR Genotyping Strategy

The screening for detection of the random integration of the transgene was achieved by PCR amplification. Two PCRs were designed (see FIG. 2):
PCR1 is designed to efficiently detect the transgene random integration event. The selected primer pair allows the amplification of a short DNA sequence within the transgene sequence, yielding a specific 486-bp PCR product.
PCR2 is designed to assess the integrity of the transgene expression cassette. The selected primer pair allows the amplification of a DNA sequence extending from 5′ region of promoter and 3′ region of Thy1 gene yielding a specific 7037-bp PCR product. As the Thy1 promoter cassette is derived from mouse genomic sequence, the PCR screen, used to investigate the integrity of the transgene expression cassette, also leads to the amplification of a 7413-bp product derived from the endogenous Thy1 gene.

TABLE 1
PCR genotyping tgisoQC transgenic mice

| | | Expected | |
|---|---|---|---|
| Primer | | product | |
| name | Primer sequence 5′-3′ | size | PCR no. |
| TgisoQC-3 | 5′-CTCGGCGTTCTACACCATT-3′ (SEQ ID NO: 3) | 486 bp | PCR1 |
| TgisoQC-4 | 5′-CTGCTCAGCTCCAGGTCA-3′ (SEQ ID NO: 4) | | |
| GX3626 | 5′-CTACAACTGGTCAGCCTGACTACTAACC-3′ | 7037 bp | PCR2 |
| GX3627 | (SEQ ID NO: 5) | | |
| | 5′-TGATCCAGGAATCTAAGGCAGCACC-3′ | | |
| | (SEQ ID NO: 6) | | |

These tests were performed to monitor the specificity of the primers and the sensitivity of the PCR reaction. Once established, these PCR conditions were used to screen the FO generation (founder animals).

TABLE 2
Protocol for genotyping tgisoQC transgenic mice by PCR1

| Reaction mix | | | Reaction step | Temp/time | Cycles |
|---|---|---|---|---|---|
| Genomic | 50 | ng | | | |
| mouse DNA | | | | | |
| Primer | 5 | pmol | denaturing | 94° C./180 s | 1x |
| dNTPs | 200 | μM | denaturing | 94° C./45 s | 35x |
| 10 x Reaction | 2.5 | μl | annealing | 58° C./60 s | 35x |
| buffer | | | | | |
| MgCl₂ | 1.5 | mM | extension | 72° C./60 s | 35x |
| Taq poly- | 1 | U | completion | 72° C./300 s | 1x |
| merase | | | | | |
| Reaction | 25 | μl | | | |
| volume | | | | | |

TABLE 3
Protocol for genotyping tgisoQC transgenic mice by PCR2

| Reaction mix | | | Reaction step | Temp/time | Cycles |
|---|---|---|---|---|---|
| Genomic | 50 | ng | | | |
| mouse DNA | | | | | |
| Primer | 0.2 | pmol | denaturing | 94° C./120 s | 1x |
| dNTPs | 350 | μM | denaturing | 94° C./15 s | 35x |
| 10 x Reaction | 2.5 | μl | annealing | 58° C./30 s | 35x |
| buffer | | | | | |
| MgCl₂ | 1.75 | mM | extension | 68° C./420 s | 35x |
| Polymerase | 1.9 | U | completion | 72° C./420 s | 1x |
| blend (Taq/Tgo | | | | | |
| polymerase) | | | | | |
| Reaction | 25 | μl | | | |
| volume | | | | | |

tgisoQC Transgenic Lines: Transgene Expression Levels

In order to prove that the generation of the transgenic animals was successful, enzymatic activity in brain homogenates of wild-type and heterozygous transgenic mice was assessed. If the strategy was successful, then a significant increase of isoQC-activity was expected. The enzyme activity was determined, applying a method, which is based on detection of formation of L-pGlu-beta-naphthylamine from L-glutaminyl-beta-naphthylamine catalyzed by isoQC or QC in cell homogenates (Cynis, H. et al. 2006 Biochim Biophys Acta 1764, 1618-1625). Briefly, the assay is based on conversion of H-Gln-βNA to pGlu-βNA. The sample consisted of 50 μM H-Gln-βNA in 25 mM MOPS, pH 7.0, 0.1 mM N-Ethylmaleinimide (NEM) and enzyme solution in a final volume of 1 ml. Substrate and NEM were pre-incubated for 15 min at 30° C. The sample was centrifuged at 4° C. for 20 min at 16,000×g. The reaction was started by addition of 100 μl brain homogenate. The reaction mix was further incubated at 30° C. and constantly shaken at 300 rpm in a thermomixer (Eppendorf, Germany). Test samples were removed at different time points of between 0 and 45 min. The reaction was immediately stopped by boiling for 4 min. Test samples were cooled on ice and stored at −20° C. For analysis, samples were thawed on ice and centrifuged at 4° C. for 20 min at 16,000×g. All HPLC measurements were performed using a RP18 LiChroCART HPLC-Cartridge and the HPLC system D-7000 (Merck-Hitachi). Briefly, 20 μl of the sample were injected and separated by increasing concentration of solvent A (acetonitrile containing 0.1% TFA) from 8% to 20% in solvent B (H₂O containing 0.1% TFA). QC activity of isoQC was quantified from a standard curve of pGlu-βNA (Bachem, Bubendorf, Switzerland) determined under assay conditions.

The results of the analysis are depicted in FIG. 8. All transgenic lines showed a significant increase of isoQC-activity in brain, suggesting an expression of the protein and a successful generation of transgenic mice. The determination was useful to detect a different degree of expression of isoQC in the mice: Highest expression rate was observed with the tg mouse line 31, the lowest expression was observed tg mouse line 20. However, even in mouse line tg isoQC 20, the activity was 10 fold higher compared to wt mice.

### Immunohistochemistry and Histology

### Methods:

For immunohistochemical staining, mice at the age of two months (tgisoQC-31 heterozygous, wildtype, and QPCTL knockout) were euthanized with carbon dioxide and transcardially perfused with washing buffer (0° C.), consisting of 137 mM NaCl, 22 mM dextrose, 23 mM sucrose, 0.2 mM CaCl₂, and 0.2 mM sodium cacodylate, pH 7.3 followed by fixation buffer, consisting of 1.3M paraformaldehyde, 0.2M sucrose, and 104 mM sodium cacodylate. Brain samples were carefully dissected and post-fixed in fixation buffer at 4° C. and embedded together in a gelatine multibrain matrix. The brains were freeze-sectioned (30 μm) using a sliding microtome. For immunohistochemistry, sections were stained free floating using the ABC method (avidin-biotin complex binding to the biotinylated secondary antibody) and DAB as substrate. For QPCTL labeling, the affinity purified polyclonal isoQC3285 antibody (Probiodrug) prepared in rabbit was used 1:1.000 as primary antibody. For neuronal labeling, the monoclonal b-NeuN antibody (AbCam) prepared in mouse was used 1:1.500 as primary antibody. For astroglia labeling, the polyclonal GFAP antibody (Dako) prepared in rabbit was used 1:50.000 as primary antibody. For microglia labeling, the polyclonal lba-1 antibody (Wako) prepared in rabbit was used 1:10.000 as antibody. For each staining the appropriate biotinylated secondary antibody was used at a dilution of 1:250.

For immunofluorescence staining, mice at the age of two months (wildtype, tgisoQC-13 heterozygous, tgisoQC-20 heterozygous, and tgisoQC-31 heterozygous) were euthanized with carbon dioxide and perfused transcardially with phosphate-buffered saline (PBS). The brains were dissected, cut into sample-pieces (˜3×5 mm), and immersion-fixated with HOPE I (hepes glutamic acid buffer mediated organic solvent protection effect) solution (over night/4° C.). The brain-pieces were incubated with HOPE II solution (2 hours/0° C.), acetone (3×2 hours/0° C.), and pre-warmed low-melting paraffin (over night/55° C.). Coronal sections of 10 μm were cut from the paraffin blocks on a sliding microtome and transferred on microscope slides. The slides were incubated in 20% formic acid for antigen retrieval. Endogenous peroxidase was inactivated with 0.5% H₂O₂ in TBS containing 0.25% Triton X 100 (10 min). Unspecific binding sites were blocked with 5% normal goat serum in TBS containing 0.25% Triton X 100 (1 h). For neuronal labeling, the monoclonal b-NeuN antibody (AbCam) prepared in mouse was used 1:2.000 as primary antibody (4° C./over night). For astroglia labeling, the polyclonal GFAP antibody (Dako) prepared in rabbit was used 1:2.000 as primary antibody (4° C./over night). As secondary antibodies Cy2 labeled goat-anti-rabbit and Cy3 labeled goat-anti-mouse were used (1:500; 1 h/RT).

### Results:

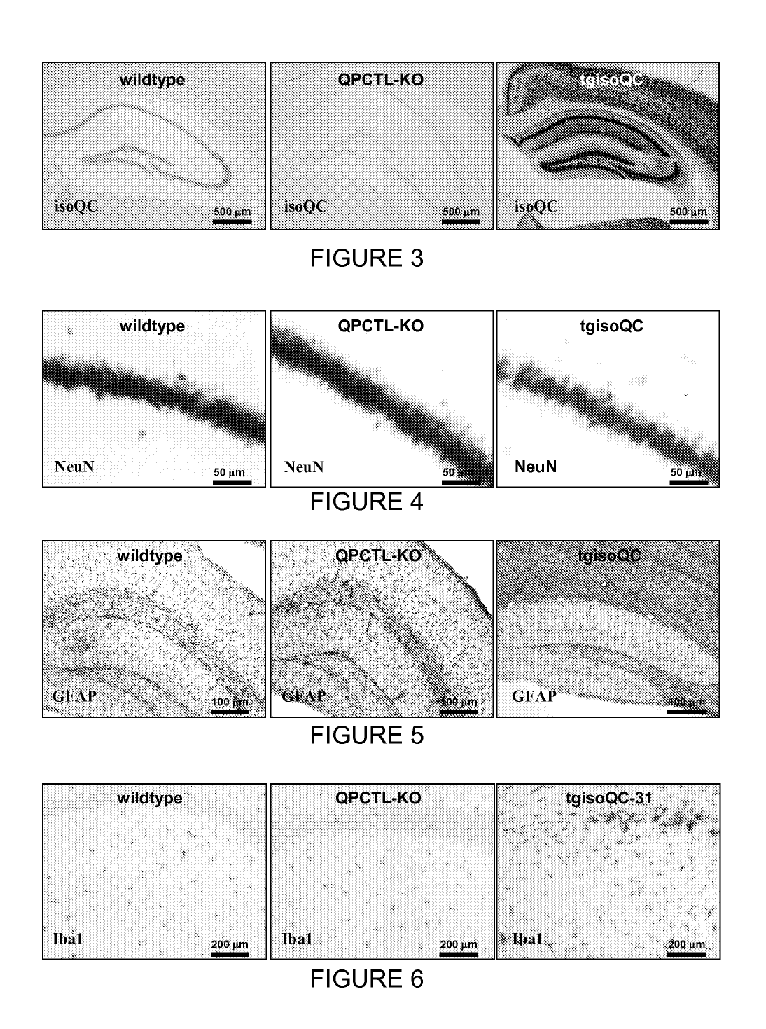

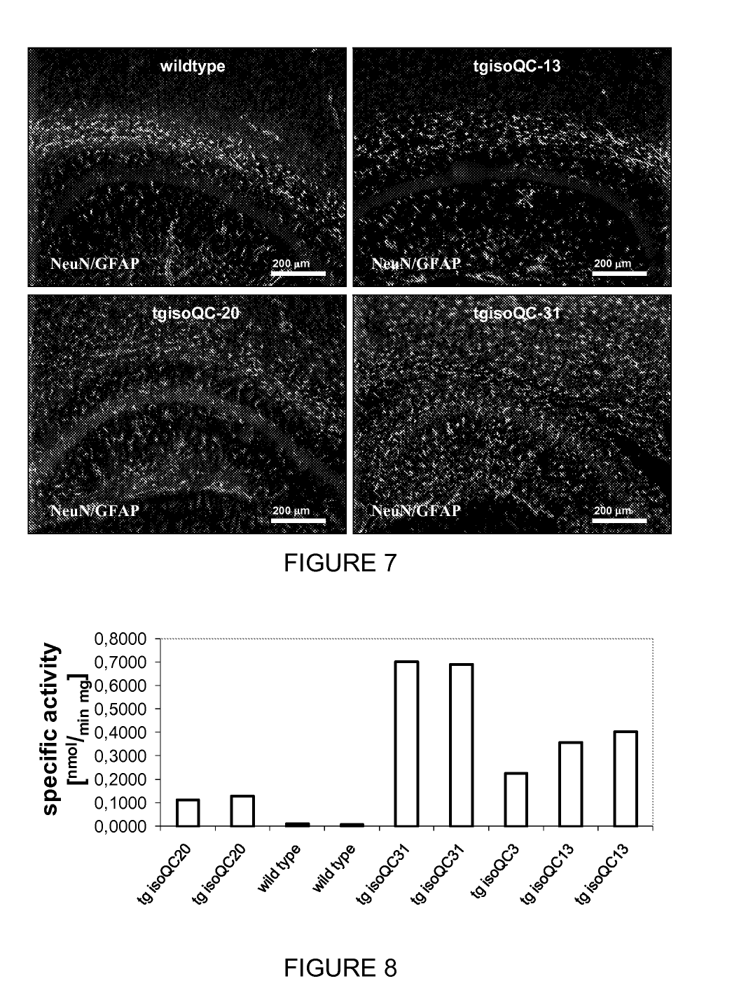

The overexpression of isoQC leads to an obvious increasing of isoQC signal in the Golgi apparatus of neurons, which is absent in QPCTL-knockout mice (FIG. 3). This overexpression leads to expression dependent neuroinflammation, visible in the hippocampus of tgisoQC mice at the age of two months (FIG. 7), which is absent at the age of one month (data not shown). The inflammation is characterized by an activation of astroglia (FIG. 5) and microglia (FIG. 6), attended by neuronal cell loss, visible in the CA1 region of the hippocampus (FIG. 4).

### Example 2

### Behavioural Tests

For behavioral characterization, a phenotyping set was generated consisting of 18 females (9 wildtype and 9 heterozygous mice). At 3 months of age these animals were investigated in a battery of 9 consecutive tests followed by short examinations in the primary screen at 4 and 6 months of age.

### Automated Home Cage Behavior Analysis

Methods: Circadian patterns of locomotor activity and ingestion behavior were assessed using a PhenoMaster system (TSE Systems, Bad Homburg, Germany). Two horizontally staked infrared-sensor frames detected locomotion in the x/y-level and rearing events in the z-level, while water and food consumption were measured by two balances. All four parameters were automatically recorded as the sum over 1 minute intervals for 136 hours (6.5 days). Experiments took place under a 12 hour light/12 hour dark cycle (lights on 06:00 h, lights off 18:00 h) and animals received water and food ad libitum in individual observation units (standard type III cages with grid lid).

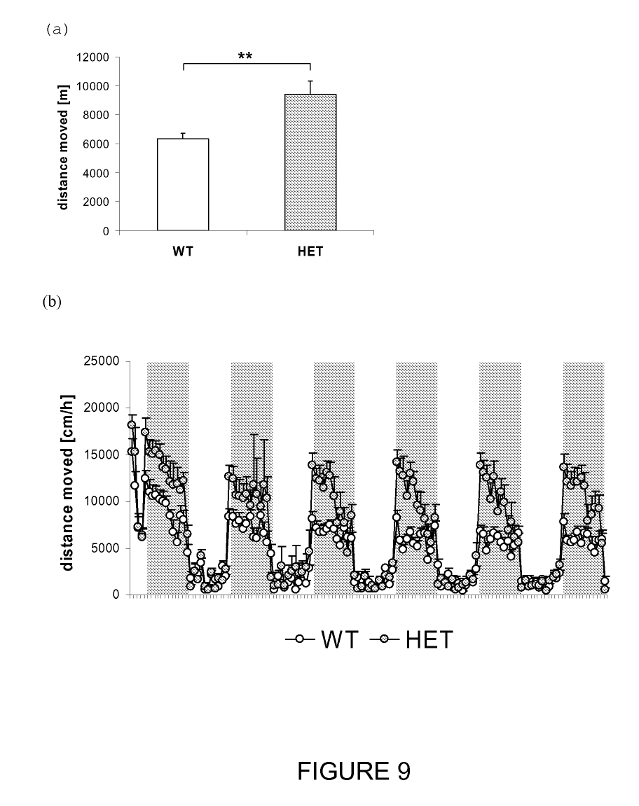

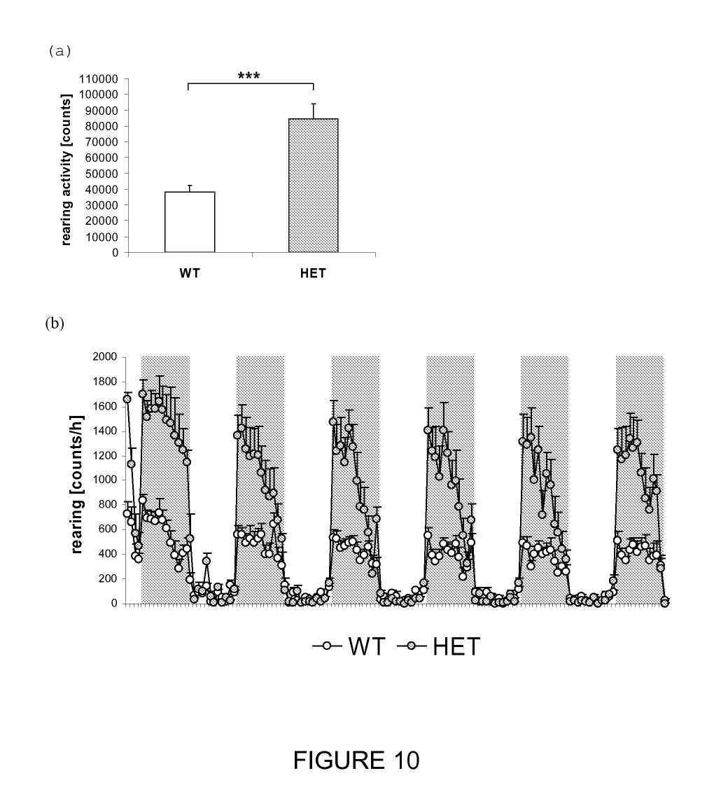

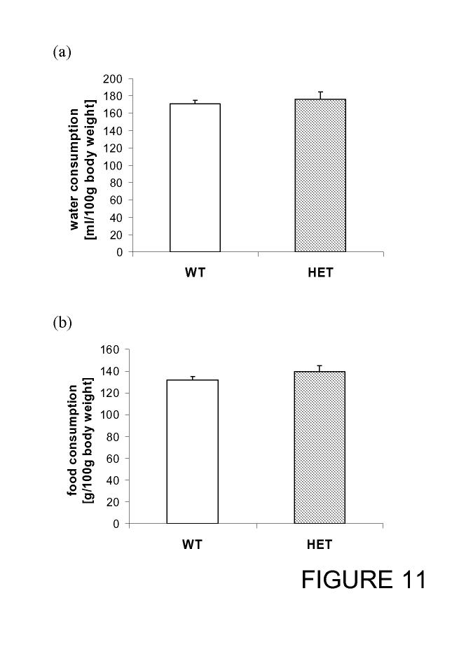

Results: Compared to wildtype animals, heterozygous tgisoQC mice displayed a 45% increase of locomotor activity in the x/y-level (FIG. 9 (a)) as well as a 120% increase of rearing activity (FIG. 10 (a)) over a 136 hour investigation period. This alteration was observed in the dark cycles (FIGS. 9 (b) and 10 (b)), in case of rearing activity also in the initial 4 hour-light phase. Analysis of ingestion behavior showed nearly identical levels of water and food consumption (FIG. 11) in both genotype groups. Circadian activity patterns also revealed no apparent shift.

### Dark-Light Box Test

Methods: Investigation of anxiety behavior was performed using the dark-light box test, which utilizes the naturalistic conflict of mice to explore novel environments and the tendency to avoid aversive open fields (Crawley J. N. (2007) What's Wrong With My Mouse: Anxiety-Related Behaviors. Wiley, Second Edition, 240-241). A dark-light box module (TSE Systems, Bad Homburg, Germany) consists of a Plexiglas chamber unequally divided into two compartments, a large (34×28 cm), open and brightly illuminated (700-1000 lux) compartment and a small (16×28 cm), closed and dark (1-2 lux) compartment, which are connected by a small alleyway. Animals were placed individually in the brightly lit arena and were allowed to freely explore both compartments for 10 minutes. The duration of stay in the light compartment served as index for the level of anxiety.

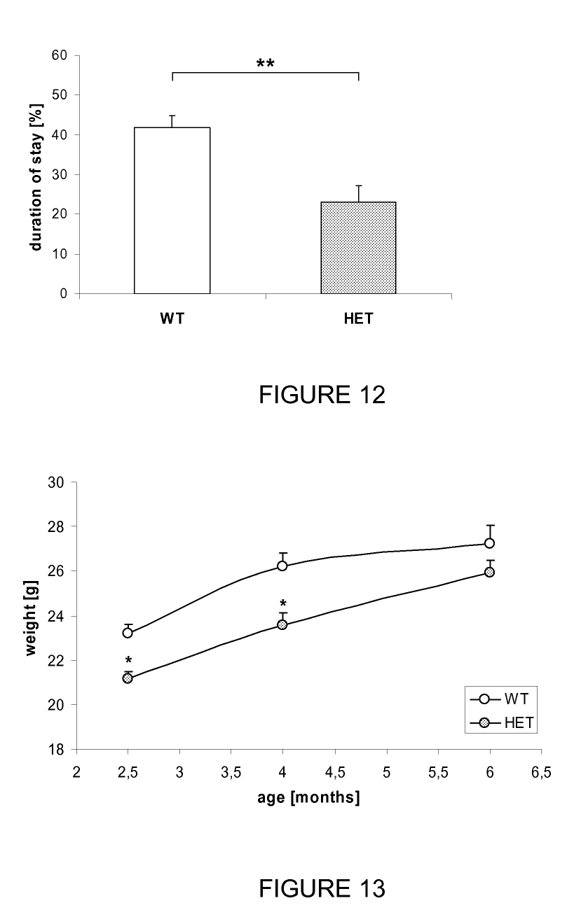

Results: Heterozygous tgisoQC mice exhibited an intensely decreased (about 40%) duration of stay in the light compartment (FIG. 12) compared to wildtype littermates.

### Primary Screen

Methods: The primary screen was used to prompt animals' general health, neurological reflexes and sensory functions (muscle and lower motor neuron functions, spinocerebellar, sensory, neuropsychiatric and autonomic functions) that could interfere with further behavioral assays. It was based on the guidelines of the SHIRPA protocol (Rogers D. C. et al., 1997. Behavioral and functional analysis of mouse phenotype: SHIRPA, a proposed protocol for comprehensive phenotype assessment. Mamm Genome, 8:711-713), which provides a behavioral and functional profile by observational assessment. The investigation started with observing social behavior in the home cage (“home cage observation”) and subsequently undisturbed behavior of single animals in a clear Plexiglas arena for 90 seconds (“individual observation”). This monitoring of mouse behavior was followed by a battery of short tests for further characterization: acoustic startle reflex, hanging behavior, visual placing, falling behavior, righting reflex, postural reflex, negative geotaxis, hanging wire, ear twitch, whiskers twitch and eye blink. At last, to complete the assessment, animals were examined for dysmorphological and weight abnormalities.

Results: Already at about 3 months of age, heterozygous tgisoQC animals displayed excited and hyperactive behavior in the primary screen. Intensive jumping in the corner of the home and observation cage as well as very fast movements in individual observation and hanging behavior were major features of this hyperactivity. Furthermore, weight was significantly reduced in HET compared to WT littermates (p<0.01, FIG. 13).

Primary screen examinations at 4 and 6 months of age revealed the same phenotypic abnormalities in heterozygous mice as seen in animals aged 3 months. Additionally cramping during hanging behavior and early falling off in the hanging wire assay could be detected in a high proportion of HET tgisoQC mice. At 6 months of age, weight differences could still be found but lost statistical significance.

### Pole Assay

Methods: The pole was used as a simple test for motor-coordinative deficits. It consists of a metal pole (diameter: 1.5 cm, length: 50 cm) wrapped with an anti-slip tape, with a plastic ball on the top, and vertically installed on a heavy platform. For testing, animals were placed head-up directly under the ball and time to orient themselves down (t-turn) and descend the length of the pole (t-total) was measured (cut-off time: 120 s). Aberrant activities (e.g. falling, jumping, sliding) were recorded as 120 s. The best performance over five trials was used for analysis.

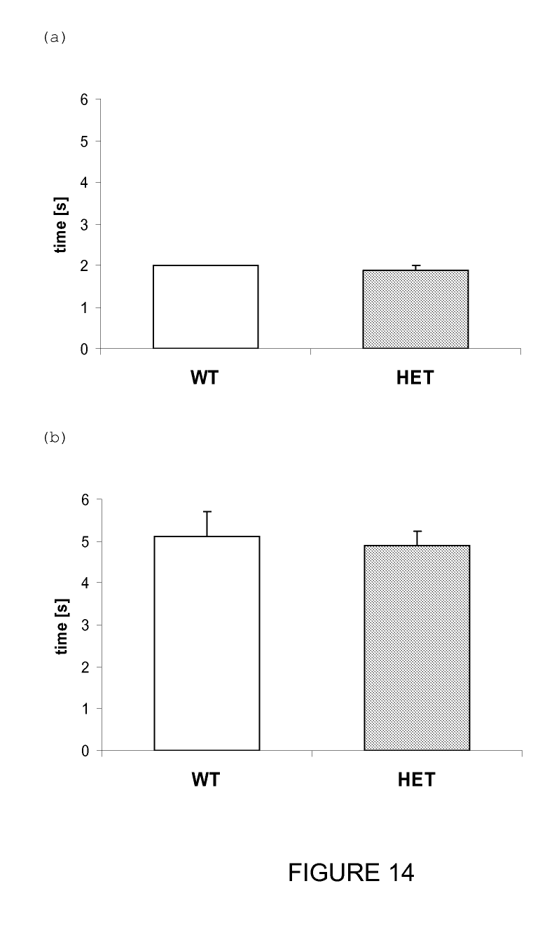

Results: Performance on the pole was comparable between both genotype groups (FIG. 14).

### Rotarod

Methods: The rotarod is a standard test widely used to investigate neuro-motor performance in rodents. It provides a quantitative assessment of coordination and balance, since animals must continuously walk forward on a horizontal, rotating cylinder to avoid falling off the rod. Testing was performed on two consecutive days, using a computer controlled RotaRod System (TSE Systems, Bad Homburg, Germany). In the first morning session, mice were trained on a constantly rotating rod (10 revolutions per minute) until they were able to stay on the drum for at least 60 seconds. In the afternoon, and on the following day, 3 test sessions were conducted, each consisting of 3 trials. The rod-speed was accelerated from 4 to 40 rpm over a five-minute period. The total distance moved until the animal fell off was calculated automatically by the system. Performance was examined for each testing trial (motor learning), and using best trial analysis (motor coordination).

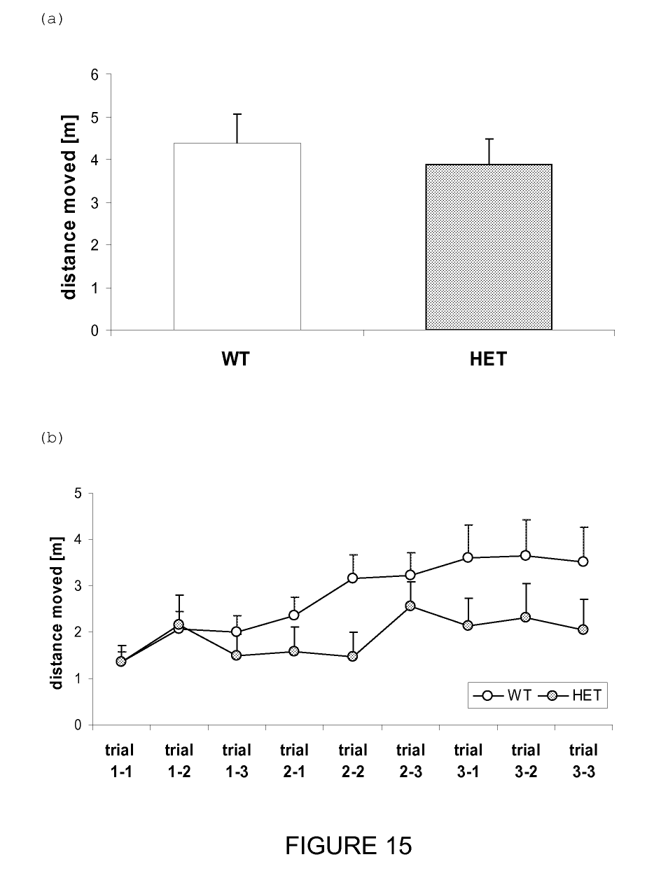

Results: Best trial analysis delivered a comparable performance between HET and WT tgisoQC females in the maximum distance moved with only a weak tendency for a reduction in HET (FIG. 15 (a)). Improvement over the nine test trials was clearly reduced in HET compared to WT littermates, but no significant differences could be found (FIG. 15 (b)).

### Holeboard Test

Methods: Mice tend to poke their noses into holes in the wall or floor. The holeboard test takes advantage of this intrinsic behavior to assess the status of exploratory behavior. Mice were placed individually into a quadratic (24×24 cm) holeboard module (TSE Systems, Bad Homburg, Germany) with 9 equally distributed holes (1.5 cm diameter) in the floor. The number of nosepokes and the total duration of hole exploration were automatically monitored for 10 minutes.

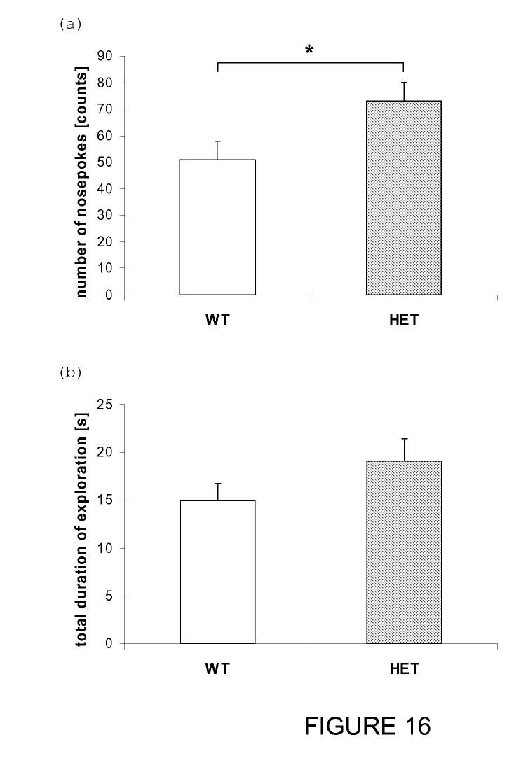

Results: The number of nosepokes was significantly increased (FIG. 16 (a)) in heterozygous tgisoQC animals compared to wildtypes and also the total duration of exploration was clearly elevated (FIG. 16 (b)).

### Tail Flick Test

Methods: The tail flick is a spinal reflex in which the mouse moves its tail out of the path of a noxious cutaneous thermal stimulus. To assess nociception, animals were tested on a TailFlick 60200 Analgesia System (TSE Systems, Bad Homburg, Germany) and tail withdrawal latency to a strong beam of focused light (circa 51° C.) was measured three times.

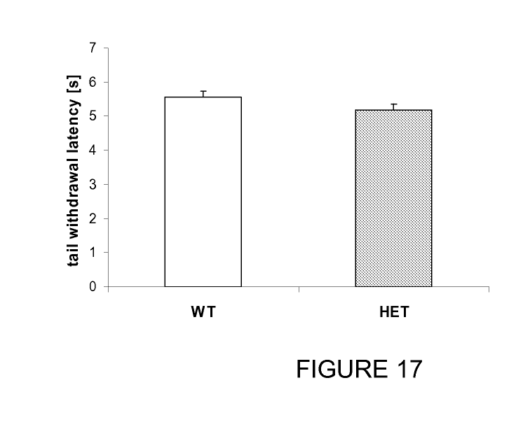

Results: Heterozygous tgisoQC mice displayed no apparent altered nociception compared to wildtype littermates (FIG. 17).

### Constant Hotplate

Methods: Tests for acute thermal pain sensitivity were performed on a constant hotplate (TSE Systems, Bad Homburg, Germany). Mice were placed in a Plexiglas cylinder on the 52.5° C. warm surface of the hotplate, and hind paw withdrawal latency (or shaking/licking of the hind paw) was measured two times (non-habituated vs. habituated). First measurements took place without former habituation. After habituation on a 32.0° C. hot plate animals were retested. Cutoff-time was 60 seconds.

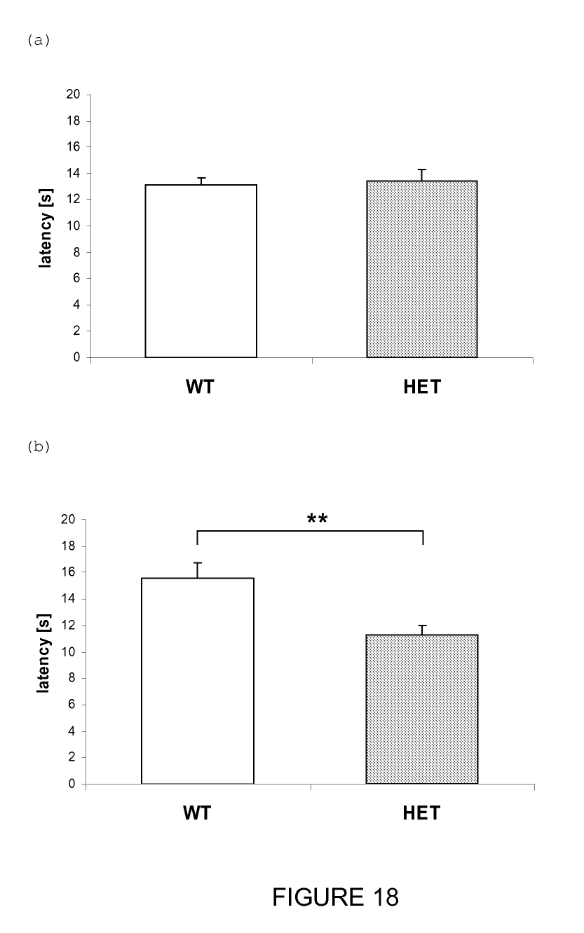

Results: In the non-habituated trial no obvious differences could be found in the hotplate performance of HET and WT tgisoQC females aged 3 months (FIG. 18 (a)). But analysis of the performance after habituating animals to the apparatus and testing procedure revealed a significant reduction of paw withdrawal latency in heterozygous animals (FIG. 18 (b)).

### Fear Conditioning

To study contextual and cued fear responses in mice a commercially available computer-controlled “Fear Conditioning System (FCS)” (TSE Systems, Bad Homburg, Germany) is used.

Experimental settings are chosen following the protocol of Oliver Stiedl (Stiedl O. et al., 2004 Behavioral and autonomic dynamics during contextual fear conditioning in mice. Auton. Neurosci. Basic and Clinical 115(1-2):15-27). Investigations in the FCS are performed on two consecutive days and are divided into three phases:

Conditioning phase (Phase 1): Conditioning is performed in a clear acrylic compartment within a constantly illuminated fear conditioning module. A loudspeaker provides a constant, white background noise. After an initial habituation period the mouse is given a defined auditory cue (conditioned stimulus), e.g. 10 kHz, 75 dB SPL for 30 s. During the end of the auditory cue a short electrical footshock (unconditioned stimulus) is administered (e.g. 0.7 mA, constant current, for 2 s). Mice are returned to their home cages 30 s after shock termination.

Contextual retention (Phase 2): 24 h after conditioning (Phase 1) animals are re-exposed to the original context and locomotor activity and freezing behavior respectively is monitored for 270 s.

Cue retention (Phase 3): Memory for the conditioned stimulus (auditory cue) is tested 1 h after Phase 2 in a novel context (similarly sized black acrylic box, reduced light intensity due to the black color, plane floor plate instead of shock grid). After 270 s of free exploration in the novel context the same auditory cue as in Phase 1 is applied for 180 s and locomotor activity and freezing behavior respectively is automatically recorded by the FCS.

### Y-Maze

Spontaneous alternation rates in the Y-Maze serve as index for spatial learning in rodents. Alternations are defined as successive entries into the three arms of a triangular Y-shaped maze in overlapping triplet sets. An entry is defined to be successive as soon as a mouse enters an arm with all four paws. The percent alternation during a 10 minute trial is automatically calculated by a “Viewer” video detecting system (Biobserve, Bonn, Germany) as the ratio of actual to possible alternations.

### Open Field

The open field test is a short test for the assessment of locomotor activity. Mice are tested using an open field module for a PhenoMaster system (TSE Systems, Bad Homburg, Germany) made of Plexiglas walls and a gray plastic floor with 50×28 cm surface area and 25 cm-high walls. Activities are automatically monitored by two horizontal staked infrared sensor frames to detect horizontal (x/y-level) and vertical activity (z-level). The behavioral parameters registered during up to 60 minute sessions are (i) distance moved in defined intervals, (ii) activity (beam breaks broken) in the central part of the arena and (iii) rearing events (the number of times an animal stood upon its hind legs with forelegs in the air or against the wall).

### Cross-Maze

The Cross-Maze consists of black plastic material (arm sizes: 30.0 cm length, 8.0 cm breadth, wall height 15.0 cm). Adjacent arms are in a 90° position. The four arms extend from a central space measuring 8.0 cm in square. Thus, the animals visit the arms via a central space. During 20.0 min test sessions, each mouse is initially randomly placed in one arm and allowed to traverse freely through the maze. Individual arms are signed 1-4. An alternation is defined as entry into four different arms on consecutive entries on overlapping quadruple sets (for example 2, 3, 4, 1 or 4, 2, 3, 1 but not 1, 2, 3, 2). An entry was defined to be successive as soon as a mouse enters an arm with all four paws. The percent alternation is calculated as the ratio of actual to overall performed alternations during the period of observation. In order to diminish odor cues, the maze was cleaned with a solution containing 30% ethanol, 60% water and 10% odorless soap after each trial. The test is being performed under modest white light conditions. Shorter timeframes for the test, i.e. 10 min, are possible.

### T-Maze Continuous Alternation Task (T-CAT)

A T-maze was used according to the measures provided by Gerlai (Gerlai, R. (1998) A new continuous alternation task in T-maze detects hippocampal dysfunction in mice. A strain comparison and lesion study. Behav Brain Res., 95, 91-101). The apparatus was made of black plastic material with a black floor and guillotine doors. Testing of the mice consisted of one single session, which started with 1 forced-choice trial, followed by 14 free-choice trials.

(i) Forced-choice trial: in the first trial, one of the two goal arms is blocked by lowering the guillotine door. After the mouse is released from the start arm, it will explore the maze, enter the open arm and return to the start position. As soon as the mouse returned to the start arm, the guillotine door was lowered and the animal was confined for 5 seconds.

(ii) Free-choice trials: After opening the door of the start arm, the animal is free to choose between both goal arms, as all guillotine doors are open. Once the mouse entered a goal arm, the other goal arm is closed. When the mouse returned to the start arm, the next free-choice trial started after 5s confinement in the start arm.

A test session was terminated after 30 min or after 14 free-choice trials were carried out. The animals were never handled during the task and the experimenter was not aware of the genotype of the tested animals. An alternation ratio was calculated for each animal by dividing the number of alternating choices by the number of total choices. Animals performing less than 8 choices in the given time frame were excluded from the analysis.

### Morris Water Maze

In the typical paradigm, a mouse is placed into a small pool of water back-end first to avoid stress, and facing the pool-side to avoid bias, which contains an escape platform hidden a few millimeters below the water surface. Visual cues, such as colored shapes, are placed around the pool in plain sight of the animal. The pool is usually 4 to 6 feet in diameter and 2 feet deep. A sidewall above the waterline prevents the mouse from being distracted by laboratory activity. When released, the mouse swims around the pool in search of an exit while various parameters are recorded, including the time spent in each quadrant of the pool, the time taken to reach the platform (latency), and total distance traveled. The mouse's escape from the water reinforces its desire to quickly find the platform, and on subsequent trials (with the platform in the same position) the mouse is able to locate the platform more rapidly. This improvement in performance occurs because the mouse has learned where the hidden platform is located relative to the conspicuous visual cues. After enough practice, a capable mouse can swim directly from any release point to the platform.

### Clasping Test

To test clasping behavior, mice were suspended by the tail for 30 sec and the hindlimb-clasping time was scored. A duration of 0 sec clasping was given a score of 0, 1-10 sec a score of 1, 10-20 sec a score of 2 and a clasping of more than 20 sec a score of 3 (Nguyen, T., Hamby, A. & Massa, S. M. (2005) Clioquinol down-regulates mutant huntingtin expression in vitro and mitigates pathology in a Huntington's disease mouse model. Proc Natl Acad Sci U.S.A., 102, 11840-11845).

### Footprint Analysis

To obtain footprints, the hindpaws were labeled with blue nontoxic ink. The animals were placed at one end of a dark tunnel (30 cm×7 cm diameter), which ends in an enclosed box. The floor of the tunnel was lined with white paper. Animals were allowed to walk to the other end of the tunnel, where they were retrieved and placed in their home cage. A minimum of two nonstop passes was required. Stride length was determined by measuring the distance between each step and average stride length was calculated (Barlow, C., Hirotsune, S., Paylor, R., Liyanage, M., Eckhaus, M., Collins, F., Shiloh, Y., Crawley, J. N., Ried, T., Tagle, D. & Wynshaw-Boris, A. (1996) Atm-deficient mice: a paradigm of ataxia telangiectasia. Cell., 86, 159-171).

### Balance Beam

Balance and general motor function were assessed using the balance beam task. A 1 cm dowel beam is attached to two support columns 44 cm above a padded surface. At either end of the 50 cm long beam a 9×15 cm escape platform is attached. The animal is placed on the center of the beam and released. Each animal is given three trials during a single day of testing. The time the animal remained on the beam is recorded and the resulting latencies to fall of all three trials are averaged. If an animal remains on the beam for the whole 60-sec trial or escapes to one of the platforms, the maximum time of 60 sec is recorded (Arendash, G. W., Gordon, M. N., Diamond, D. M., Austin, L. A., Hatcher, J. M., Jantzen, P., DiCarlo, G., Wilcock, D. & Morgan, D. (2001) Behavioral assessment of Alzheimer's transgenic mice following long-term Abeta vaccination: task specificity and correlations between Abeta deposition and spatial memory. DNA Cell Biol., 20, 737-744).

### String Suspension Task

As a test of agility and grip strength, a 3 mm cotton string is suspended 35 cm above a padded surface in the beam apparatus. The animals are permitted to grasp the string by their forepaws and are released. A rating system from 0 to 5 is used during the single 60-sec trial to assess each animals' performance in this task: 0=unable to remain on the string; 1=hangs only by fore- or hindpaws; 2=as for 1, but attempts to climb onto string; 3=sits on string and is able to hold balance; 4=four paws and tail around string with lateral movement; 5=escape (Moran, P. M., Higgins, L. S., Cordell, B. & Moser, P. C. (1995) Age-related learning deficits in transgenic mice expressing the 751-amino acid isoform of human beta-amyloid precursor protein. Proc Natl Acad Sci USA, 92, 5341-5345).

### Vertical Grip Hanging Task

Animals were tested for neuromuscular abnormalities (balance and muscle strength) by suspending them from wire bars (40×20 cm area with 1 mm wires 1 cm apart). Latency to fall within 60 sec was measured after a mouse was placed on the bars and turned upside down (height 30 cm) (Erbel-Sieler, C., Dudley, C., Zhou, Y., Wu, X., Estill, S. J., Han, T., Diaz-Arrastia, R., Brunskill, E. W., Potter, S. S. & McKnight, S. L. (2004) Behavioral and regulatory abnormalities in mice deficient in the NPAS1 and NPAS3 transcription factors. Proc Natl Acad Sci USA., 101, 13648-13653).

### Forced Swimming Test

The forced swimming test is performed in an identical manner to a probe test in the Morris Water Maze (Spittaels, K., Van den Haute, C., Van Dorpe, J., Bruynseels, K., Vandezande, K., Laenen, I., Geerts, H., Mercken, M., Sciot, R., Van Lommel, A., Loos, R. & Van Leuven, F. (1999) Prominent axonopathy in the brain and spinal cord of transgenic mice overexpressing four-repeat human tau protein. Am J Pathol, 155, 2153-2165). In brief, a pool with a diameter of 110 cm is filled with opaque water to a height of 20 cm and is kept at 22° C. The mice were placed in the middle of the pool for one 60-sec single trial and total swimming distance and swimming speed were measured using a computer automated tracking system (VideoMot2, TSE-Systems).

### Elevated Plus-Maze

The Elevated Plus-Maze was built according to the description of Lister (1987). It had a black Plexiglas floor with a 5×5 cm central square platform, from which radiated two 45×5 cm open arms with 0.25 cm high edges and two 45×5 cm closed arms with 40 cm high walls made of clear Plexiglas. A white line was drawn half way along each of the four arms so as to measure locomotion. The apparatus was raised to 45 cm above the floor on a plus-shaped plywood base. The apparatus was located in a 2×5 m laboratory room that was illuminated with a 60-watt red light bulb.

Procedure: Mice were carried into the test room in their home cages. Mice were handled by the base of their tails at all times. Mice were placed, one at a time, in the central square of the Plus-Maze facing an open arm. The mice were then allowed to explore the apparatus for 5 minutes. An observer sitting quietly about 1 m from the apparatus recorded the behaviour of the animals on the maze. A video camcorder located 150 cm above the center of the maze also recorded behaviour. Behaviours were scored using Limelight. After 5 minutes, mice were removed from the maze by the base of their tails and returned to their home cage.

The maze was then cleaned with a solution of 70% ethyl alcohol and permitted to dry between tests.

Behaviours scored included:
1. Open arms entries: Frequency with which the animal entered the open arms. All four of the mouse's paws were required to be in the arm to be counted as an entry.
2. Closed arm entries: Frequency with which the animal entered the closed arms. All four of the mouse's paws were required to be in the arm to be counted as an entry.
3. Open arm duration: Length of time the animal spent in the open arms.
4. Closed arm duration: Length of time the animal spent in the closed arms.
5. Center square entries: Frequency with which the animal entered the central square with all four paws.
6. Central square duration: Length of time the animal spent in the central square.
7. Head dipping: Frequency with which the animal lowered the head over the sides of the open arm toward the floor.
8. Stretch attend postures: Frequency with which the animal demonstrates forward elongation of head and shoulders followed by retraction to original position.
9. Rearing: Frequency with which the animal stands on hind legs or leans against walls of the maze with front paws.
10. Nonexploratory behaviour: Grooming or any time the mouse is not moving.
11. Urination: Number of puddles or streaks of urine.
12. Defecation: Number of fecal boli produced.
13. Locomotion: Number of times the animal crossed a line drawn on the open and closed arms.

From these results, the percentage of entries into the open arms and closed arms based on the total arms entries were calculated for each animal. The percentage of time spent in the open arms and the closed arms was calculated over the 5 minute test. The index of open arm avoidance (Trullas, R., & Skolnick, P. 1993. Differences in fear motivated behaviors among inbred mouse strains. Psychopharmacology, 111, 323-331) was calculated as [100−(% time on open arms+% entries into the open arms)\2].

What is claimed is:

1. A transgenic non-human animal for overexpressing isoQC, comprising cells containing a DNA transgene encoding human isoQC, characterized in that said human isoQC comprises the amino acid sequence of SEQ ID NO: 1 or an amino acid sequence having at least 75% sequence identity to the amino acid sequence of SEQ ID NO: 1 or a fragment or derivative of the amino acid sequence of SEQ ID NO: 1.

2. The transgenic non-human animal of claim 1, wherein the human isoQC consists of the amino acid sequence of SEQ ID NO: 1.

3. The transgenic non-human animal of claim 1, wherein the DNA transgene comprises the nucleotide sequence of SEQ ID NO: 2 or substantially the same nucleotide sequence of SEQ ID NO: 2.

4. The transgenic non-human animal of claim 1, wherein the DNA transgene consists of the nucleotide sequence of SEQ ID NO: 2.

5. The transgenic non-human animal of claim 1, wherein the animal is heterozygous for the transgene.

6. The transgenic non-human animal of claim 1, wherein the animal is homozygous for the transgene.

7. The transgenic non-human animal of claim 1, wherein the animal is a mouse.

8. The transgenic non-human animal of claim 1, wherein the transgene is operably linked to a tissue-specific promoter.

9. The transgenic non-human animal of claim 1, having two or more of the following features:
the human isoQC consists of the amino acid sequence of SEQ ID NO: 1;
the DNA transgene comprises the nucleotide sequence of SEQ ID NO: 2 or substantially the same nucleotide sequence of SEQ ID NO: 2;
the DNA transgene consists of the nucleotide sequence of SEQ ID NO: 2;
the animal is heterozygous for the transgene;
the animal is homozygous for the transgene;
the animal is a mouse; or
the transgene is operably linked to a tissue-specific promoter.

10. A method of screening for biologically active agents that inhibit or promote isoQC production in vivo, comprising:
administering a test agent to the transgenic non-human animal of claim 1; and
determining the effect of the agent on the amount of isoQC produced.

11. A cell or cell line derived from the transgenic non-human animal according to claim 1.

12. A transgenic mouse comprising a transgenic nucleotide sequence encoding isoQC, which comprises the nucleotide sequence of SEQ ID NO: 2 or substantially the same nucleotide sequence of SEQ ID NO: 2, operably linked to a promoter, integrated into the genome of the mouse, wherein the mouse demonstrates a phenotype that can be reversed or ameliorated with an isoQC inhibitor.

13. A method of screening for therapeutic agents that inhibit or promote isoQC activity comprising:
(a) administering test agents to the transgenic mouse of claim 12;
(b) evaluating the effects of the test agent on the neurological phenotype of the mouse; and
(c) selecting a test agent which inhibits or promotes isoQC activity.

14. A method of the treatment or prevention of an isoQC-related disease or a QC-related disease comprising:
(a) administering the selected test agent of claim 13; and
(b) monitoring the patient for a decreased clinical index for an isoQC-related disease or a QC-related disease.

15. The method of claim 14, wherein the isoQC-related disease or the QC-related disease is selected from the group consisting of: mild cognitive impairment, Alzheimer's disease, Familial British Dementia, Familial Danish Dementia, neurodegeneration in Down Syndrome, Huntington's disease, Kennedy's disease, ulcer disease, duodenal cancer with or w/o Helicobacter pylori infections, colorectal cancer, Zolliger-Ellison syndrome, gastric cancer with or without Helicobacter pylori infections, pathogenic psychotic conditions, schizophrenia, infertility, neoplasia, inflammatory host responses, cancer, malign metastasis, melanoma, psoriasis, rheumatoid arthritis, atherosclerosis, pancreatitis, restenosis, lung fibrosis, liver fibrosis, renal fibrosis, graft rejection, acquired immune deficiency syndrome, impaired humoral and cell-mediated immune responses, leukocyte adhesion and migration processes in the endothelium, impaired food intake, impaired sleep-wakefulness, impaired homeostatic regulation of energy metabolism, impaired autonomic function, impaired hormonal balance or impaired regulation of body fluids, multiple sclerosis, the Guillain-Barré syndrome and chronic inflammatory demyelinizing polyradiculoneuropathy.

16. A method of investigation of the physiological function of isoQC comprising:
(a) Crossbreeding of the isoQC transgenic non-human animals of claim 1 with a non-human animal model, which is specific for a desired disease,
(b) Breeding and ageing the crossbred animals and the disease specific animals;
(c) Monitoring the disease state age-dependently in the crossbred animals,
(d) As a control group, monitoring the disease state age-dependently in the disease specific animal models that are not transgenic for isoQC,
(e) Calculating the differences in the disease state in the crossbred animals versus the disease specific animals, and
(f) Determining the effect of the isoQC transgene on the disease state.

17. The method of claim 16, comprising one or more of the following features:
wherein the crossbred animals are heterozygous for the isoQC transgene;
wherein the crossbred animals are homozygous for the isoQC transgene;
wherein the recombinant isoQC, which is overexpressed in the crossbred non-human animals, leads to one or more of an earlier outbreak of the specific disease, an accelerated course of the specific disease or a more severe course of the specific disease;
wherein the recombinant isoQC leads to the increase or decrease of the level of one or more isoQC substrates in the crossbred non-human animals;
wherein the disease specific animal model is selected from PDAPP, Tg2576, APP23, TgCRND8, PSEN1M146V or PSEN1M146L, PSAPP, APPDutch, BRI-Aβ40 and BRI-Aβ42, JNPL3, TauP301S, TauV337M, TauR406W, rTg4510, Htau, TAPP and 3×TgAD;
wherein the isoQC substrate is selected from [Glu3]Aβ3-40/42/43 or [Glu11]Aβ11-40/42/43;
wherein the disease specific animal model is the apoE deficient mouse; or
wherein the recombinant isoQC leads to the increase or decrease of the level of one or more isoQC substrates in the crossbred non-human animals and the isoQC substrate is a chemokine selected from CCL2, CCL8, CCL7, CCL13, CCL 16, and CCL 18.

18. A method of screening for activity decreasing effectors of isoQC comprising:
(a) Crossbreeding of the isoQC transgenic non-human animals of claim 1 with a non-human animal model, which is specific for a desired disease,
(b) Administering a test agent to a treatment group of crossbred animals,
(c) Administering a placebo to a control group of crossbred animals,
(d) Monitoring the disease state age-dependently in the treatment group,
(e) Monitoring the disease state age-dependently in the control group,
(f) Calculating the differences in the disease state in the treatment group versus the control group, and
(g) Determining the effect of the test agent on the disease state.

19. The method of claim 18, comprising one or more of the following features:
wherein the crossbred animals are heterozygous for the isoQC transgene;
wherein the crossbred animals are homozygous for the isoQC transgene;
wherein the recombinant isoQC, which is overexpressed in the crossbred non-human animals, leads to one or more of an earlier outbreak of the specific disease, an accelerated course of the specific disease or a more severe course of the specific disease;
wherein the recombinant isoQC leads to the increase or decrease of the level of one or more isoQC substrates in the crossbred non-human animals;
wherein the disease specific animal model is selected from PDAPP, Tg2576, APP23, TgCRND8, PSEN1M146V or PSEN1M146L, PSAPP, APPDutch, BRI-Aβ40 and BRI-Aβ42, JNPL3, TauP301S, TauV337M, TauR406W, rTg4510, Htau, TAPP and 3×TgAD;
wherein the recombinant isoQC leads to the increase or decrease of the level of one or more isoQC substrates in the crossbred non-human animals and the isoQC substrate is selected from [Glu3]Aβ3-40/42/43 or [Glu11]Aβ11-40/42/43;
wherein the disease specific animal model is the apoE deficient mouse; or
wherein the recombinant isoQC leads to the increase or decrease of the level of one or more isoQC substrates in the crossbred non-human animals and the isoQC substrate is a chemokine selected from CCL2, CCL8, CCL7, CCL13, CCL 16, and CCL 18.

20. A pharmaceutical composition comprising the selected test agent as defined in claim 13.
